(19) United States
(12) Patent Application Publication
(10) Pub. No.: US 2019/0209650 A1
(43) Pub. Date: Jul. 11, 2019

(54) METHOD TO IMPROVE THE EFFECTIVENESS OF ANTI-CANCER THERAPIES BY EXPOSING CANCER CELLS TO AN INFLAMMATORY STIMULUS PRIOR TO TREATMENT

(71) Applicants: William Warren HARLESS, Middle River (CA); Myron SZEWCZUK, Kingston (CA)

(72) Inventors: William Warren HARLESS, Middle River (CA); Myron SZEWCZUK, Kingston (CA)

(21) Appl. No.: 16/312,841

(22) PCT Filed: Jun. 22, 2017
(86) PCT No.: PCT/CA2017/050765

§ 371 Date: Dec. 21, 2018

Related U.S. Application Data
(60) Provisional application No. 62/464,199, filed on Feb. 27, 2017.
(60) Provisional application No. 62/353,446, filed on Jun. 22, 2016.

(51) Int. Cl. A61K 38/18; A61K 38/20; A61P 35/04; C07K 14/52

(52) U.S. Cl. CPC A61K 38/1833; A61K 38/1858; C07K 14/52; A61P 35/04; A61K 38/204

(57) ABSTRACT

There is provided methods and compositions for treating cancer or sensitizing cancer to chemotherapy in a patient by administering at least one cytokine prior to chemotherapy treatment.

This application claims the benefit of U.S. Provisional Application No. 62/353,446 filed Jun. 22, 2016 and U.S. Provisional Applications No. 62/464,199 filed Feb. 27, 2017.

### FIELD OF THE INVENTION

The present invention relates to a method for treatment of patients with cancer, and more specifically to with a method of improving the effectiveness of chemotherapy treatment.

### BACKGROUND OF THE INVENTION

Cancers of epithelial origin account for 90% of cancer deaths worldwide.

The standard treatment of most potentially curable solid tumors is surgical removal often followed by chemotherapy. For the major cancer killers such as lung, breast, and colorectal cancer, the administration of chemotherapy after the tumour is surgically removed may eradicate micrometastatic disease (disease undetectable using conventional imaging technologies) in those patients who still harbor residual cancer cells after surgery. However, this treatment is often unsuccessful.

The resistance of any given cancer cell to conventional medical treatments may not primarily result from the possession or acquisition of specific point mutations but instead largely reside in a distinct cancer cell subpopulation of cancer stem cells. In addition to being relatively resistant to conventional medical therapies, cancer stem cells are also capable of metastasis and tissue colonization. As few as 200 cancer cells displaying the stem cell phenotype can form tumours in animal models, while 20,000 cancer cells without the stem cell phenotype fail to form tumours. These cells are therefore particularly relevant to cancer metastasis and recurrence and treatment resistance.

Cancers of various types respond to chemotherapy very differently. Some cancers are very sensitive to the effects of chemotherapy agents (germ cell tumors, high grade lymphomas, triple negative breast cancers); while many cancers are relatively resistant to chemotherapy treatments, particularly curative chemotherapy treatments. Part of the reason for this resistance is due to the intrinsic nature of the biology of the various cancer cells, something for which there is little control over at present.

Thus, there is a need for new therapeutic strategies in treating patients with cancer.

### SUMMARY OF THE INVENTION

In one aspect, there is provided a composition for treating cancer comprising at least one cytokine that induces the cancer cell population to proliferate and/or differentiate for administration within 48+/−8 hours of a chemotherapy treatment.

In another aspect, the at least one cytokine is administered within 24+/−8 hours of a chemotherapy treatment. In another aspect, the at least one cytokine is administered within 12+/−8 hours of a chemotherapy treatment.

In another aspect, the composition for treating cancer comprises treatment with at least one cytokine administered prior to commencement of the chemotherapy treatment.

In another aspect, the at least one cytokine is administered at most 72 to 48 hours prior to chemotherapy treatment.

In another aspect, the at least one cytokine is administered 48+/−8 hours prior to chemotherapy treatment. In another aspect, the at least one cytokine is administered 24+/−8 hours prior to chemotherapy treatment. In yet another aspect, the at least one cytokine is administered 12+/−8 hours prior to chemotherapy treatment.

In another aspect, the chemotherapy treatment is discontinued between about 48 and 96 hours following treatment with the at least one cytokine.

In one aspect, the at least one cytokine is HGF.

In another aspect at least one cytokine is HGF and Il-6.

In another aspect, the cytokine administered is one or more of Il-6, PDGF-BB, HGF.

In one aspect, the chemotherapy treatment comprises a neu-1 sialidase inhibitor. In one embodiment, the neu-1 sialidase inhibitor is oseltamivir phosphate.

In one aspect, the chemotherapy treatment further comprises a non-steroidal anti-inflammatory drug (NSAID). In one embodiment, the NSAID is aspirin.

In one aspect, the chemotherapy treatment further comprises a biguanide. In one embodiment, the biguanide is metformin.

In one aspect, the chemotherapy treatment comprises a checkpoint inhibitor.

In one aspect, there is provided a method of treating cancer comprising administering to a patient in need thereof the composition described herein. In one aspect there is provided use of the composition described herein for the treatment of cancer.

In one aspect, there is provided a method of treating cancer comprising administering to a patient in need thereof the composition as described herein as adjuvant therapy. In one aspect there is provided use of the composition described herein as adjuvant therapy for the treatment of cancer.

In one aspect, there is provided a method of sensitizing cancer cells to chemotherapy, comprising administering to a patient in need thereof the composition as described herein. In one aspect there is provided use of the composition described herein for sensitizing cancer cells to chemotherapy.

In one aspect, there is provided a method of preventing or reducing cancer metastasis, recurrence, or chemotherapy resistance, the method comprising administering to a patient in need thereof the composition as described herein. In one aspect there is provided use of the composition described herein for preventing or reducing cancer metastasis, recurrence, or chemotherapy resistance.

### BRIEF DESCRIPTION OF THE FIGURES

FIGS. 1A-1H depicts scatter plots showing the percentage of EpCAM positive cells and Cancer Stem Cell (CSC) Subpopulation (CD44+CD133-) after incubation with Cytokines over a period of 0 hrs.

FIGS. 2A-2H depicts scatter plots showing the percentage of EpCAM positive cells and Cancer Stem Cell (CSC) Subpopulation (CD44+CD133-) after incubation with Cytokines over a period of 24 hrs.

FIGS. 3A-3H depicts scatter plots showing the percentage of EpCAM positive cells and Cancer Stem Cell (CSC) Subpopulation (CD44+CD133-) after incubation with Cytokines over a period of 48 hrs.

FIG. 4 depicts a table showing percentage of Cancer Stem Cells remaining after exposure to various cytokines and 24 hours exposure to CPT-11 (Irinotecan).

FIG. 5 depicts tables showing cancer cell viability after 12 hour HGF and IL-6 pretreatment, followed by 72 hour combination treatment. A) MDAMB231 TmxR 12 Hr cytokine exposure followed by 72 Hr of combination treatment (COMBO=30 μM paclitaxel, 250 μg/mL oseltamivir phosphate, and 2.5 mM aspirin). B) MDAMB231 TmxR 12 Hr IL-6+HGF exposure followed by 72 Hr of combination treatment (COMBO=50 μM paclitaxel, 250 μg/mL oseltamivir phosphate, and 2.5 mM aspirin). HI1=HGF 0.5 ng/ml+IL-6 50 ng/ml. HI2=HGF 1 ng/ml+IL-6 100 ng/ml. HI3=HGF 5 ng/ml+IL-6 500 ng/ml.

FIG. 6 depicts a table showing cancer cell viability of MDAMB231 cell lines in TmxR media after 24 hour HGF and IL-6 pretreatment, followed by 72 hour combination treatment (COMBO=30 μM paclitaxel, 250 μg/mL oseltamivir phosphate, and 2.5 mM aspirin).

FIG. 7 depicts tables showing cancer cell viability after 48 hours of HGF only pretreatment, followed by 72 hour combination treatment. HI1=HGF 0.5 ng/ml; H12=HGF 1 ng/ml; HI3=HGF 5 ng/ml. A) PANC1 48 hour HGF exposure followed by 72 hour combination treatment (COMBO=30 paclitaxel, 250 μg/mL oseltamivir phosphate, and 2.5 mM aspirin). B) PANC1 48 hour HGF exposure followed by 72 hour combination treatment (COMBO=50 μM paclitaxel, 250 μg/mL oseltamivir phosphate, and 2.5 mM aspirin).

FIG. 8 depicts a table showing cancer cell viability after 72 hours of HGF only pretreatment, followed by 72 hour combination treatment (COMBO=30 μM paclitaxel, 250 μs/mL oseltamivir phosphate, and 2.5 mM aspirin). HI1=HGF 0.5 ng/ml; HI2=HGF 1 ng/ml; HI3=HGF 5 ng/ml.

### DETAILED DESCRIPTION

In the following description, numerous specific details are set forth to provide a thorough understanding of the invention. However, it is understood that the invention may be practiced without these specific details.

### Cancer Stem Cells and Metastatic Cancer

In contrast to the proven ability of chemotherapy to cure micrometastatic cancer in some patients, clinically evident metastatic cancer is generally incurable. Given the emerging evidence of the importance of cancer stem cells in drug resistance and metastatic efficiency, the eradication of this cancer cell subpopulation may be critical to achieve cancer cure.

Differentiated cancer cells may dedifferentiate to a cancer stem cell phenotype, either spontaneously, or, after certain triggering mechanisms. After an initial treatment against cancer, such as surgery, chemotherapy, or radiation, tissue damage induced by that treatment will trigger the release of specific inflammatory molecules fostering the induction of a partial epithelial-mesenchymal transition (EMT) in the remnant cancer cell population and reversion to a cancer stem cell phenotype. The present inventor provides evidence that these same signaling pathways foster cancer stem cell self-renewal as a highly conserved response to tissue damage. The net result of this process is the rapid emergence of a stem cell enriched residual cancer cell population.

EMT induction confers a migratory and invasive epithelial phenotype critical to tissue repair; stem cell activation provides the epithelial cell precursors necessary to regenerate tissue. A rapid, even transient, phenotypic shift in cancer cells to a more migratory, metastatic phenotype in response to a treatment induced release of tissue repair signals may have significant clinical implications. Cancer cell populations that are enriched for cancer stem cells are highly resistant to ionizing radiation, conventional chemotherapy, and highly tumorigenic.

The term “cancer”, as used herein, may mean a malignant neoplasm that has undergone characteristic anaplasia with loss of differentiation, increased rate of growth, invasion of surrounding tissue, and is capable of metastasis. Residual cancer is cancer that remains in a subject after any form of treatment given to the subject to reduce or eradicate a cancer and recurrent cancer is cancer that recurs after such treatment. Metastatic cancer is a cancer at one or more sites in the body other than the site of origin of the original (primary) cancer from which the metastatic cancer is derived. In the case of a metastatic cancer originating from a solid tumor, one or more (for example, many) additional tumor masses can be present at sites near or distant to the site of the original tumor.

In an aspect, the cancer originates from a solid tumour.

The term “tumor”, as used herein, refers to a neoplasm or an abnormal mass of tissue that is not inflammatory, which arises from cells of pre-existent tissue. A tumor can be either benign (noncancerous) or malignant (cancerous). Tumors can be solid or hematological. Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelogenous leukemia, and chronic lymphocytic leukemia), myelodysplastic syndrome, and myelodysplasia, polycythemia vera, lymphoma, (such as Hodgkin's disease, all forms of non-Hodgkin's lymphoma), multiple myeloma, Waldenstrom's macroglobulinemia, and heavy chain disease. Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, lung cancer, ovarian cancer, prostate cancer, benign prostatic hyperplasia, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, epithelial tumors (e.g., cervical cancer, gastric cancer, skin cancer, head and neck tumors), testicular tumor, bladder carcinoma, melanoma, brain tumors, and CNS tumors (such as a glioma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma).

In some aspects, the tumour is a malignant solid tumour.

As used herein, the term “metastasis” refers to the growth of a cancerous tumor in an organ or body part, which is not directly connected to the organ of the original cancerous tumor. Metastasis may be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, or primary tumour, and migration and/or invasion of cancer cells to other parts of the body.

In some aspects, metastasis refers to the subsequent growth or appearance of a cancerous tumour in a different location to an original tumour after treatment of the original tumour.

As used herein, the term “recurrence” refers to further growth of neoplastic or cancerous cells after diagnosis of cancer or a primary tumour. Particularly, recurrence may occur when further cancerous cell growth occurs in the cancerous tissue at the site of the original cancer. The cancer may come back to the same place as the original cancer/primary tumor or to another place in the body.

In some aspects, recurrence refers to a cancer that has reappeared at the site of an original cancer or primary tumour after treatment of that original cancer or primary tumour, after a period of time during which the cancer or tumour could not be detected.

The term “treatment” as used herein generally means obtaining a desired physiologic effect. The effect may be prophylactic in terms of completely or partially preventing a disease or condition or symptom thereof and/or may be therapeutic in terms of a partial or complete cure for an injury, disease or condition and/or amelioration of an adverse effect attributable to the injury, disease or condition and includes arresting the development or causing regression of a disease or condition.

“Cancer stem cells”, as used herein, are defined and functionally characterized as a small subset of cells from a tumor that can grow indefinitely in vitro under appropriate conditions (i.e., possess the ability for self-renewal), and are able to form tumors in vivo using only a small number of cells. Other common approaches to characterize cancer stem cells involve morphology and examination of cell surface markers, transcriptional profile, and drug response.

“Stem cell enrichment”, as used herein, means the increase in size or proportion or concentration of a population of cancer stem cells locally at the site of a cancer or tumour in a patient or in a location distal to the cancer or tumour. Stem cell enrichment may, in some aspects, include cancer stem-cell self-renewal, partial or complete induction of EMT in a cancer cell, or cancer stem cell proliferation.

### Methods, Compounds and Compositions

In one aspect, there is provided methods and compositions for treating a cancer in a patient.

As used herein in one embodiment, a cancer patient refers to a mammal with cancer, in one embodiment, a human patient diagnosed with cancer.

As used herein, “chemotherapy” is defined as a type of cancer treatment that uses chemical agents or drugs to destroy cancer cells. Chemotherapeutic agents include agents that target the machinery of cell division. Chemotherapy agents can include cytotoxic chemotherapy. Such chemotherapeutic agents may be selected from, but are not limited to, alkylators (including busulphan/carmustine/carboplatin/chlorambucil/cyclophosphamide/cisplatin/dacarbazine/estramustine/lomustine/melphalan/thiotepa/treosulphan); cytotoxic antibiotics, such as anthracyclines (including doxorubicin/idarubicin/epirubicin/aclarubicin/mitozantrone); topoisomerase inhibitors 1 and 2 (including doxorubicin/irinotecan/etoposide/topotecan); taxanes (including docetaxel/paclitaxel/abraxane); vinca alkaloids (including vincristine/vinblastine/vindesine/vinorelbine); and antimetabolites (including 5-FU/Gemcitabine/cladribine/Cytarabine/fludarabine/mercaptopurine/methotexate/Pemetrexed/pentostatin/t ioguanine).

Chemotherapeutic agents can also include immunotherapies. For example, blockers of the Programmed Death PD-1/PD-L1 pathway can be effective treatment modalities for a variety of cancers in a minority of patients who are eligible for treatment with these novel therapeutic compounds (Sharma et al., 2015). The checkpoint signaling pathway involves the programmed death 1 (PD-1) receptor and its ligands (PD-L1/2). This pathway is critical in triggering immune suppression of cytotoxic T cells and thereby preventing immune destruction of cancer cells. Blocking this pathway, either the receptor or its ligands by using checkpoint inhibitors, makes antitumor responses from cytotoxic T-lymphocytes more likely, thereby providing a basis for developing chemotherapeutic immunotherapies. However, challenges chemotherapeutic immunotherapies include, only a minority of patients will respond to these therapies, and those who respond initially will often develop resistance after initial response. Resistance to these therapies can be primary or acquired. Primary resistance refers to initial resistance to these therapies; acquired resistance is the development of resistance after responding initially to the treatments. The biological mechanisms that underlie resistance to these novel immunotherapies are the subject of research, hence finding ways to make tumors more susceptible to potential immunotherapy treatments represent greater chemotherapy options for cancer patients.

Examples of checkpoint inhibitors include but are not limited to: ipilimumab, pembrolizumab, nivolumab, atezolizumab, avelumab, and durvalumab.

As used herein, “a chemotherapy treatment” is used to refer to administration of one or more chemotherapeutic agents at a distinct time point, which may be administered as a single dose or may be administered as multiple doses, which may be sequential, for a period which may extend up to a few days, in one embodiment, up to 96 hours, after which there is typically a rest period during which a patient is not administered chemotherapeutic agents. In one embodiment, the rest period after treatment with chemotherapy is one week or more. In some embodiments, the rest period after treatment with chemotherapy is 7+/−3 days, 14+/−3 days, 21+/−3 days, or 28+/−3 days. In one embodiment, the rest period is one week. In some embodiments, such as where strong chemotherapy treatment is administered, the rest period after treatment is two weeks or more to allow for bone marrow recovery. In one embodiment, the rest period is at least 14 to 21 days, where strong treatment is administered.

For some chemotherapy treatments, such as metronomic chemotherapy, treatment is administered continuously on a daily basis without a rest period and at low doses of oral chemotherapy drugs.

One reason for resistance to current chemotherapy treatment is that the cancer cells that are generally most sensitive to chemotherapy agents are the cells that are most rapidly dividing, which renders them sensitive to the effects of chemotherapy drugs that often target cancer cells by disrupting the process of cell division.

Cancerous tumor resistance to chemotherapy is thought to be due to a specific type of cancer cell—the cancer stem cell. Cancer stem cells are thought to be resistant to the effects of chemotherapy and radiation through various mechanisms, including the upregulated expression of a multidrug resistance phenotype. However, another reason cancer stem cells may be resistant to the effects of conventional anti-cancer therapies is that these cells are often dormant and not dividing.

If cancer cells can be induced to proliferate and/or differentiate from an immature stem cell-like phenotype to a more differentiated phenotype shortly prior to, simultaneous to, or shortly after treatment with chemotherapy (these time windows being detailed further below), it may be possible to render these cells more vulnerable to these therapies, and thereby improve our ability to treat the disease more effectively. In preferred embodiments, cancer cells are induced to proliferate and/or differentiate from an immature stem cell-like phenotype to a more differentiated phenotype prior to treatment with chemotherapy.

The present inventor has identified that cancer cells in culture and isolated from patients can be induced to proliferate by exposure to a combination of several distinct cytokines. Distinct cytokines include, but not necessarily limited to, Hepatocyte Growth Factor (HGF), Interleukin 6 (IL-6), prostaglandin E2 (PGE-2), Monocyte Chemoattractant Protein 1 (MCP-1), Matrix metallopeptidase 9 (MMP-9), transforming growth factor beta (TGF-beta), Platelet derived growth factor BB (PDGF-BB) and placental growth factor (PGF). In some embodiments, cancer cells in culture and isolated from patients can be induced to proliferate by exposure to a combination of cytokines including, Il-6, PDGF-BB, TGF-beta, and Hepatocyte Growth Factor (HGF).

In various embodiments, the combination of cytokines includes, at least one, at least two, at least three, at least four at least five, at least six, at least seven, or all of, Hepatocyte Growth Factor (HGF), IL-6, PGE-2, MCP-1, MMP-9, TGF-beta, PDGF-BB, and PGF. In one embodiment, the combination of cytokines includes, at least one, at least two, at least three, or all of Il-6, PDGF-BB, TGF-beta, and HGF. In some embodiments, the combination of cytokines is HGF, or HGF and Il-6.

Exposing cancer cells in culture to these cytokines fosters a substantial increase in the percentage of cells that are proliferating versus comparable cell populations not exposed to these cytokines. This proliferation is induced by about 24 hours+/−8 hours after exposure to these cytokines. This increase in proliferation is seen in both the cancer stem cell and more differentiated cancer cell populations.

In one embodiment, a method for treating cancer is provided comprising administering to a patient in need thereof at least one cytokine that induces the cancer cell population to proliferate and/or differentiate within 72, 60, 48, 42, 36, 30, 24, 18, 12, or 6 hours of commencement of a chemotherapy treatment, and preferably within 48, 24, or 12 hours of chemotherapy treatment (and in one embodiment not more than 72, 60, 48, 42, 36, 30, 24, 18, 12 or 6 hours prior to commencement of the chemotherapy treatment). In some embodiments, exposure to the at least one cytokine is discontinued within 48 hours to 72 hours of chemotherapy treatment. In other embodiments, exposure to the at least one cytokine is discontinued within 48 hours to 36 hours of chemotherapy treatment.

In one embodiment, there is provided a method for treating cancer comprising administering to a patient in need thereof at least one cytokine that induces the cancer cell population to proliferate and/or differentiate prior to chemotherapy treatment. In some embodiments, at least one cytokine is administered within 72, 60, 48, 42, 36, 30, 24, 18, 12, or 6 hours prior to chemotherapy treatment, preferably 48 hours, 24 hours, or 12 hours prior to chemotherapy treatment. In some embodiments, at least one cytokine is administered up to 48 hours to 72 hours prior to chemotherapy treatment. In other embodiments, at least one cytokine is administered up to 48 hours to 36 hours prior to chemotherapy treatment.

In some embodiments, the chemotherapy treatment is administered for a few hours. In some embodiments, the chemotherapy treatment is administered for 5 minutes, 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, or 12 hours. In some embodiments, the chemotherapy treatment is administered for a period of up to 48 hours, 60 hours, or 72 hours. In some embodiments, the chemotherapy treatment is administered for 72 hours by continuous infusion.

In some embodiments, the chemotherapy treatment is discontinued between about 48 hours (+/−8 hours) and 96 hours (+/−8 hours) after it is commenced. In some embodiments, chemotherapy treatment is administered as described above following treatment with the at least one cytokines to sensitize the cancer to chemotherapy.

In another embodiment, administration of at least one cytokine together with chemotherapy treatment may be repeated in the form of recurring treatment cycles with or without a period of rest in between. In some embodiment, the treatment may be repeated as needed.

The chemotherapy treatment may be a combination treatment. In one embodiment, chemotherapy treatment comprises a neu-1 sialidase inhibitor, preferably oseltamivir phosphate. Optionally, the treatment may further comprise a non-steroidal anti-inflammatory drug (NSAID), preferably aspirin. Optionally, the treatment may further comprise a biguanide, preferably metformin.

In one aspect, the chemotherapy treatment includes drugs that disrupt the influence of the acute inflammatory response on a surviving cancer cell population. These drugs act to prevent, disrupt, or ameliorate the downstream effects of tissue repair signaling cascades induced by treatments that damage a cancerous tumor. These tissue repair cascades can serve to facilitate regrowth of a cancerous tumor by activating stem cell self-renewal and EMT. These processes in turn will facilitate regrowth of the cancer and a stem cell enriched residual cancer cell population.

In some embodiments the chemotherapy treatment is or includes a neu-1 sialidase inhibitor such as oseltamivir phosphate and analogues thereof that prevent the dimerization of receptors involved in tissue repair signaling cascades and hence prevent downstream activation. In another embodiment, the chemotherapy treatment is or includes a small molecule inhibitor of the transcriptional activators triggered by ligand-receptor interactions of receptors involved in tissue repair signaling cascades, including inhibitors of transcriptional activators such as NF-kb and Stat-3, among others. Other therapeutic agents that may be employed to disrupt the effects of these ligand receptor interactions are miRNA therapeutics that disrupt the post-transcriptional activity of target genes upregulated by these ligand receptor interactions.

In one aspect, the chemotherapy treatment includes drugs that are inhibitors of platelet aggregation and activation, which have the potential to interfere with tumour cell-platelet interactions. Non-steroidal anti-inflammatory drugs (NSAIDs) inhibit cyclooxygenase-1 (COX-1), thereby reducing platelet release of thromboxane and subsequent platelet activation, are the most commonly-used drugs affecting this pathway. Accordingly, these drugs may be used to prevent, and reverse, platelet activation and thus platelet-tumour cell adhesion during the perioperative period.

In some embodiments the chemotherapy treatment is or includes a NSAIDs that are COX-1 and/or COX-2 inhibitor. In some embodiments the chemotherapy treatment is or includes ketorolac, flurbiprofen, suprofen, ketoprofen, indometacin, aspirin, naproxen, tolmetin, ibuprofen, ampyrone, fenoprofen, zomepirac, niflumic acid, sodium salicilate, diflunisal, piroxicam, tomoxiprol, meclofenamate, sulindac, diclofenac, nimesulide, celecoxib, meloxicam, etodaloc, or rofecoxib. In some embodiments the chemotherapy treatment is or includes a COX-1 selective inhibitor. In preferred embodiments the chemotherapy treatment is or includes aspirin.

In some embodiments the chemotherapy treatment is or includes a biguanide, such as metformin and phenformin. In preferred embodiments, the chemotherapy treatment is or includes metformin.

The effectiveness of an anti-cancer treatment such as a chemotherapeutic drug may, for example, be time and concentration dependent. This may be due to the drug being removed from the body through physiological processes such as hepatic or renal clearance. The time frame a given drug dosage is effective may vary, requiring additional dosages over time to maintain therapeutically effective concentrations in the patient undergoing treatment. As some chemotherapeutics may have a narrow therapeutic index (i.e., dose limiting toxicity may be found at levels necessary for therapeutic effectiveness), daily dosing of chemotherapy may not be possible over an extended period of time.

As used herein, “therapeutically effective amount” refers to an amount effective, at dosages and for a particular period of time necessary, to achieve the desired therapeutic result. A therapeutically effective amount of a pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

In the case of administration of a cytokine or combination of cytokines as described herein, a “therapeutically effective amount” is an amount that achieves sufficient serum level or concentration of cytokine or combination of cytokines in a patient that will induce some or all the cancer stem cells to proliferate and/or differentiate from an immature stem-cell-like phenotype to a more differentiated phenotype, or that will sensitize the cancer to chemotherapy.

In some cases, a therapeutically effective amount can be determined on a case by case basis e.g. by obtaining a sample from a patient and then testing various cytokine concentrations in the sample to determine dose or readjust dose for subsequent administration.

In one embodiment, the HGF serum levels are about 0.1 ng/ml to 50 ng/ml, about 1 ng/ml to long/ml, preferably about 4 ng/ml; while the IL-6 serum levels are about 0.1 ng/ml to 500 ng/ml, about 0.1 ng/ml to 50 ng/ml, about 0.1 ng/ml to 1 ng/ml, preferably about 0.3 ng/ml to about 0.5 ng/ml to have a sensitizing effect on a cancerous tumor.

In some embodiments, serum level or concentration of HGF is at least 0.1 ng/ml, at least 0.5 ng/ml, at least 1 ng/ml, at least 5 ng/ml, or at least 10 ng/ml; preferably the concentration of HGF is 0.5 ng/ml, 1 ng/ml, or 5 ng/ml. In some embodiments, concentration of IL-6 is at least 1 ng/ml, at least 10 ng/ml, at least 50 ng/ml, at least 100 ng/ml, or at least 500 ng/ml; preferably the concentration of IL-6 is 50 ng/ml, 100 ng/ml, or 500 ng/ml. In the case of HGF and IL-6 combination treatments, various combinations of the above serum level or concentrations are achieved. In preferred embodiments of HGF and IL-6 combinations, the concentrations are: 0.5 ng/ml HGF and 50 ng/ml IL-6; 1 ng/ml HGF+100 ng/ml IL-6; or 5 ng/ml HGF and 500 ng/ml IL-6.

As used herein, “therapeutic agent” means any chemical or biological material, and may be a compound or composition, suitable for administration by methods known to those in the art, which induces a desired biological or pharmacological effect. The effect may be local or it may be systemic.

The therapeutic agents as described herein may be administered in combination with one or more pharmaceutically acceptable carriers. As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Pharmaceutically acceptable carriers must be compatible with both the components of the composition and the patient. Other examples of non-aqueous solvents include propylene glycol and other glycols, metabolizable oils, aqueous carriers including water and alcoholic/aqueous solutions, and emulsions or suspensions (eg. saline and buffered media).

Suitable dosage ranges of chemotherapeutic agents are reported in the literature and may be readily ascertained by those of skill in the art.

Embodiments described above are used in the treatment of cancer. For example, embodiments described herein can be used as adjuvant therapy to chemotherapy. Some embodiments may be used in sensitizing cancer cells to chemotherapy; while other embodiments may be used in enhancing the effects, efficacy, or potency of chemotherapy.

Embodiments described above may also be used in preventing or reducing cancer metastasis. Some embodiments may be used in preventing or reducing cancer recurrence; while other embodiments may be used in preventing or reducing cancer chemotherapy resistance.

Sources of cytokines include, for example, Millipore™ or Sigma-Aldrich™. Other sources of cytokines are also available.

### Example 1

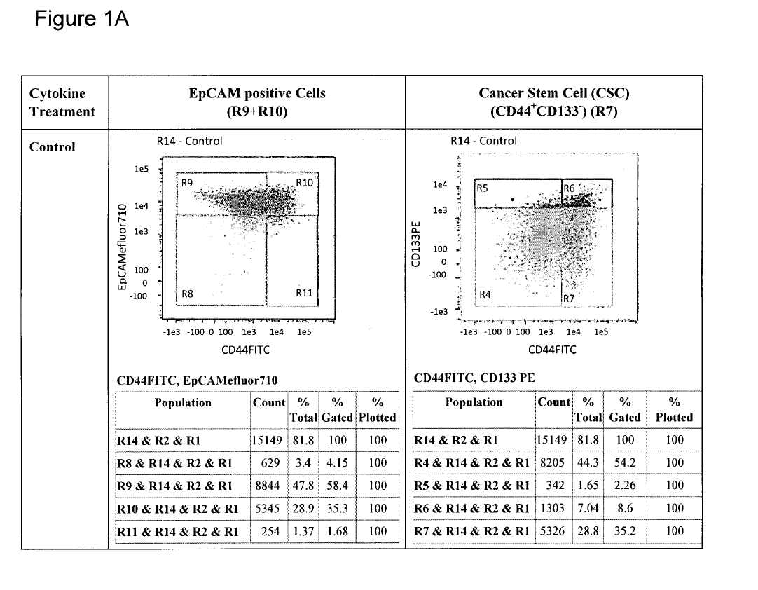

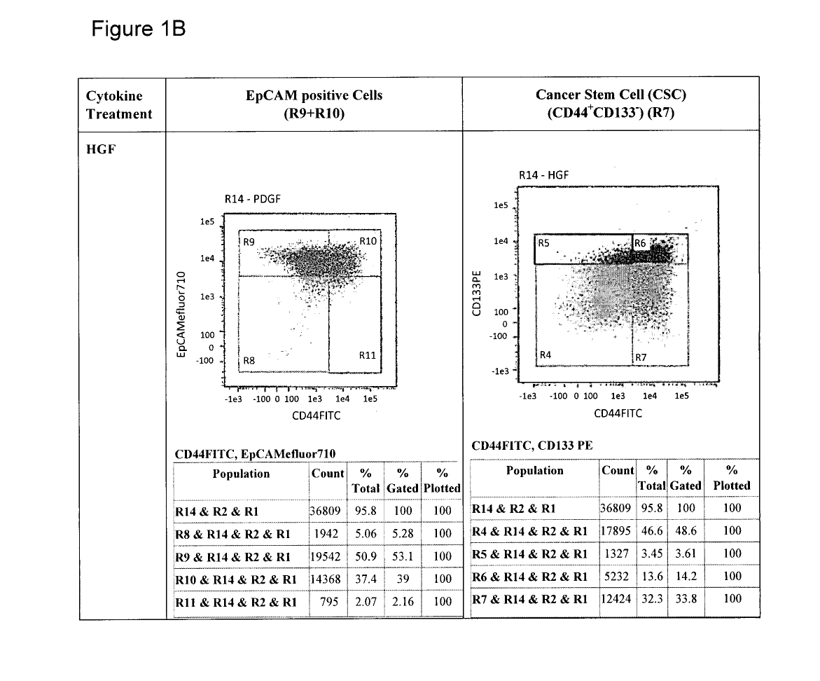

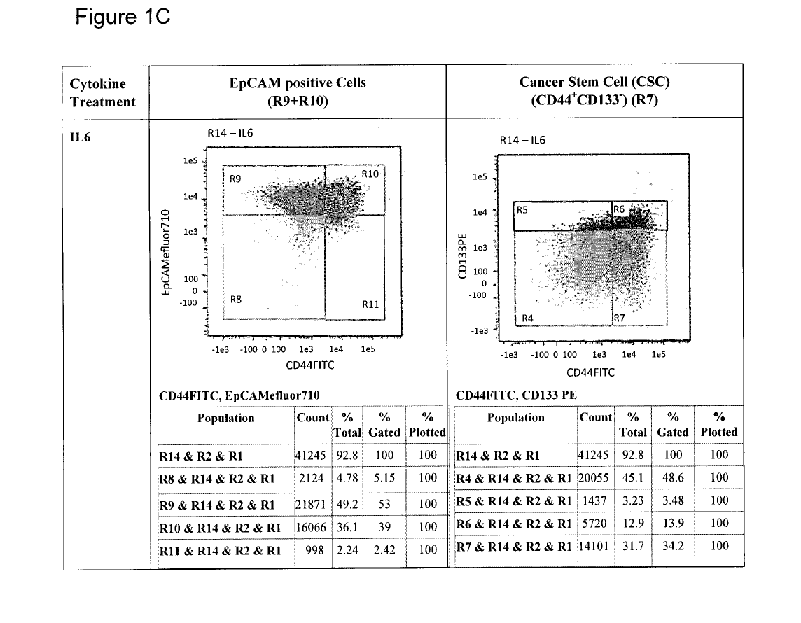

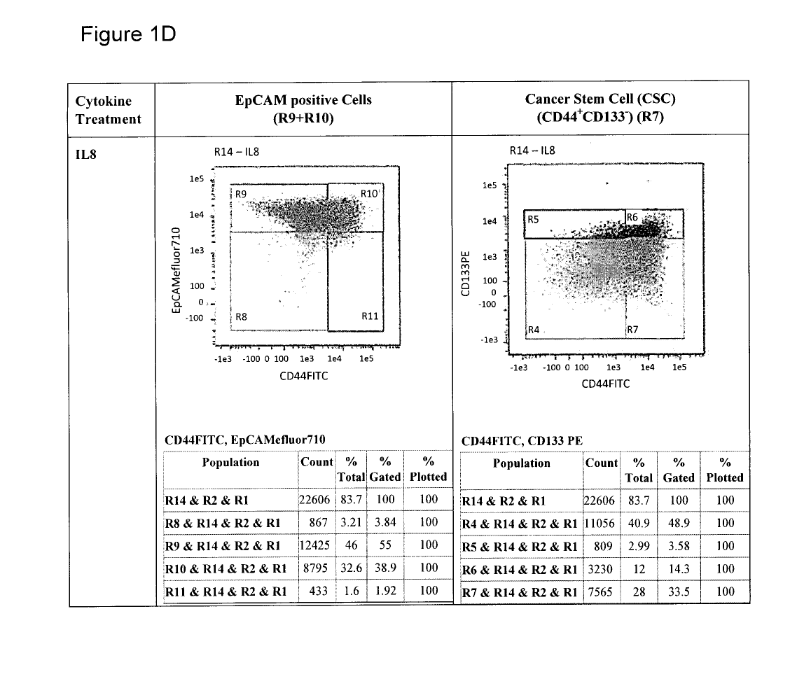

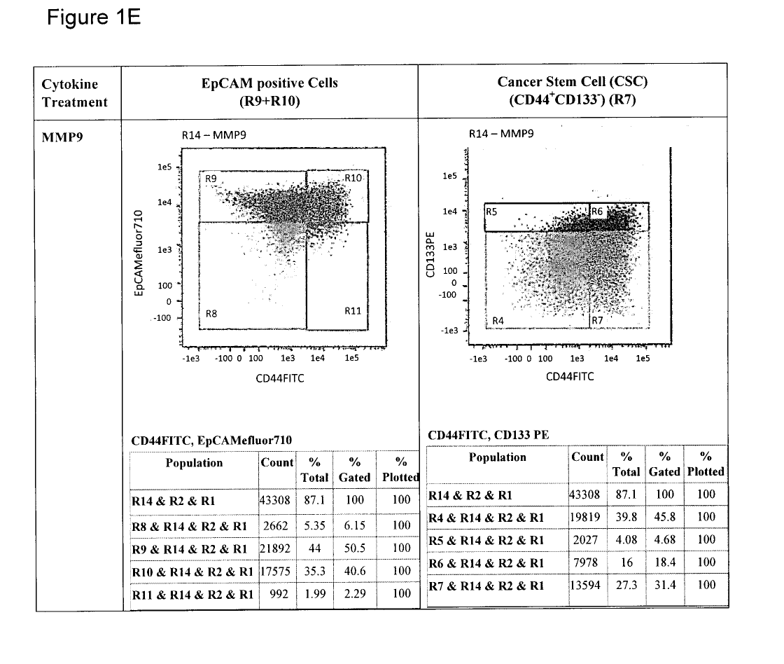

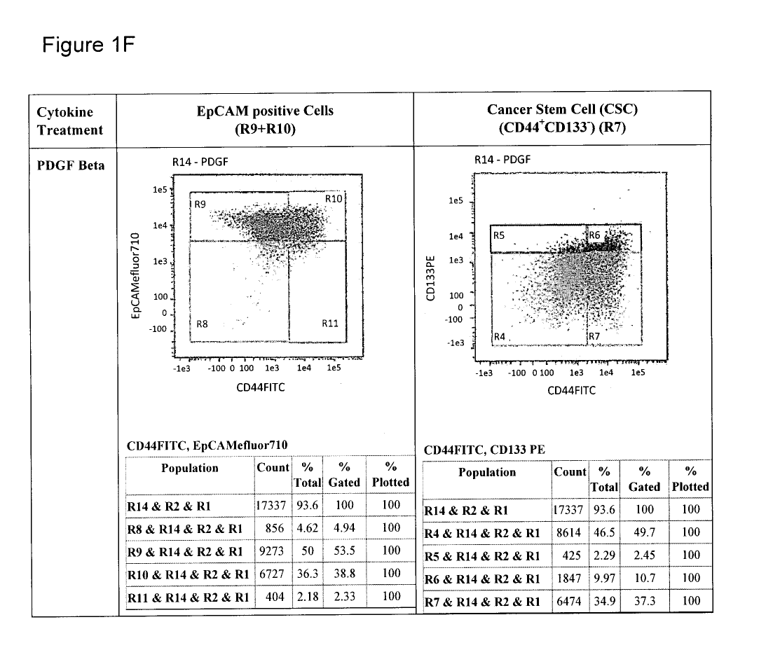

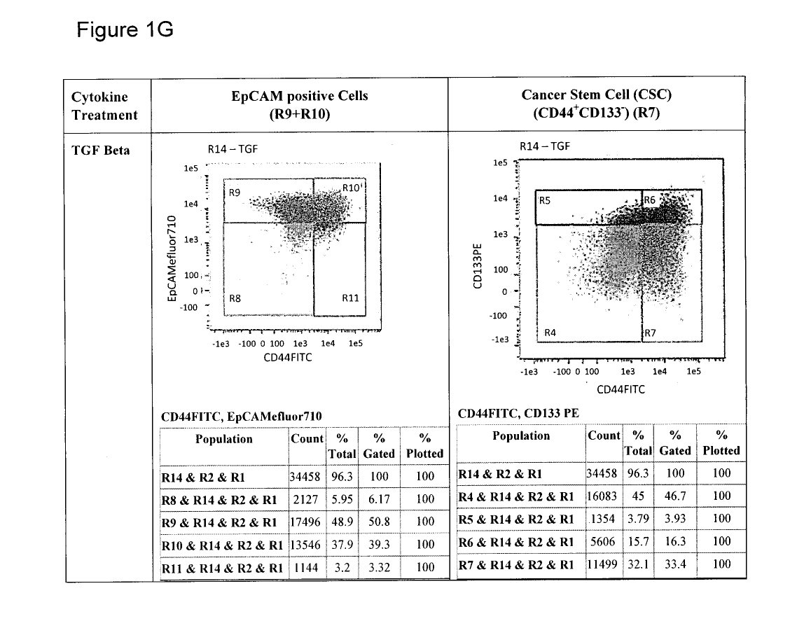

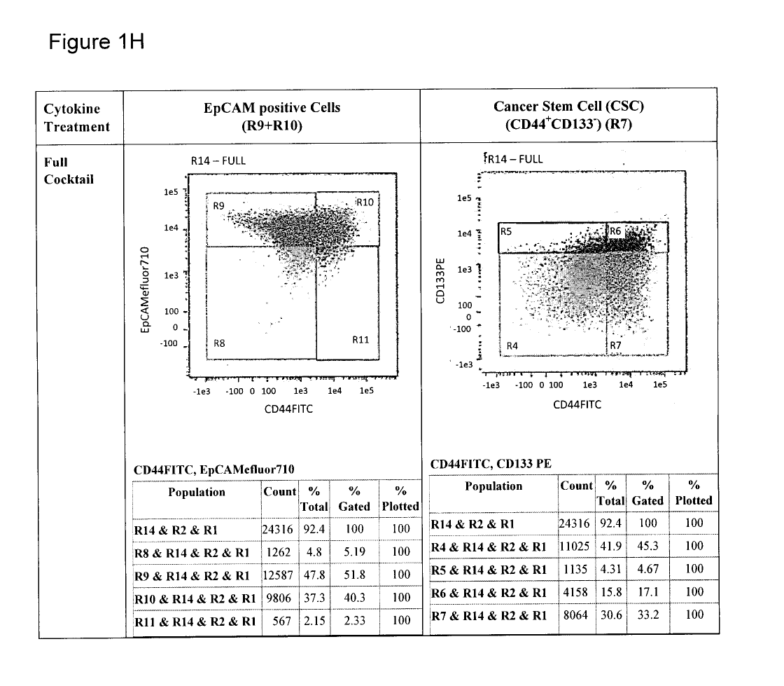

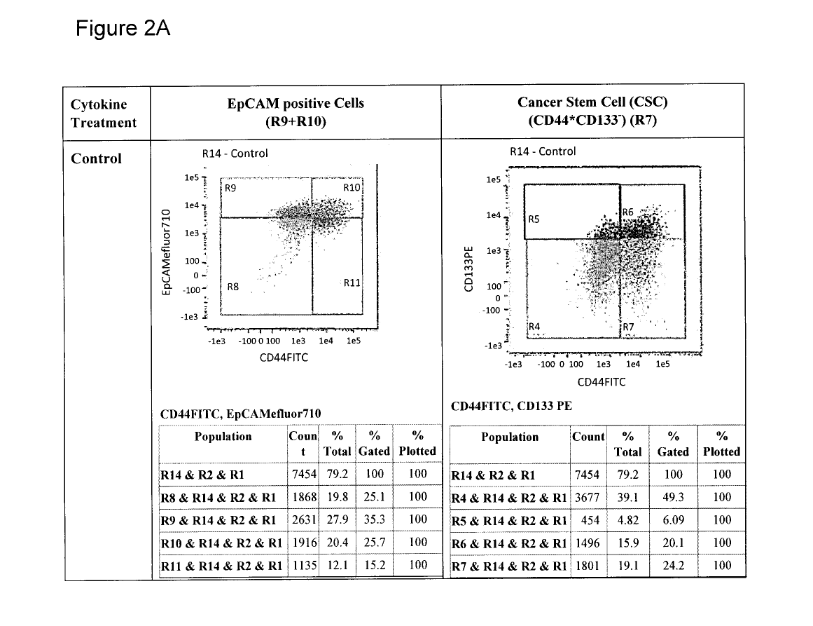

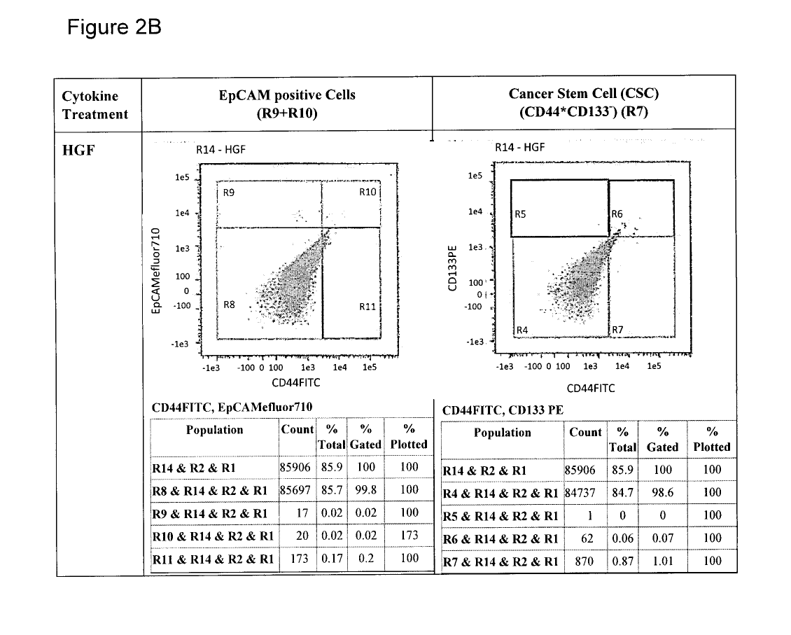

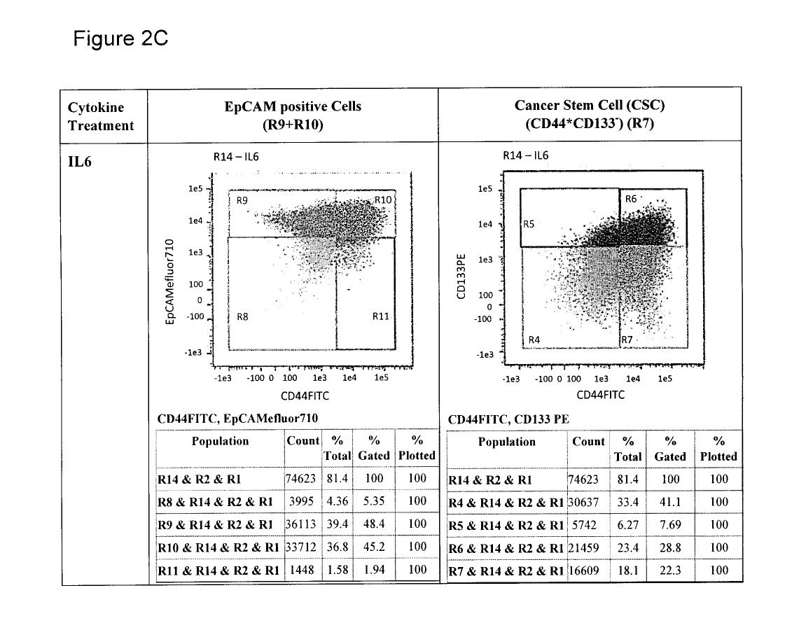

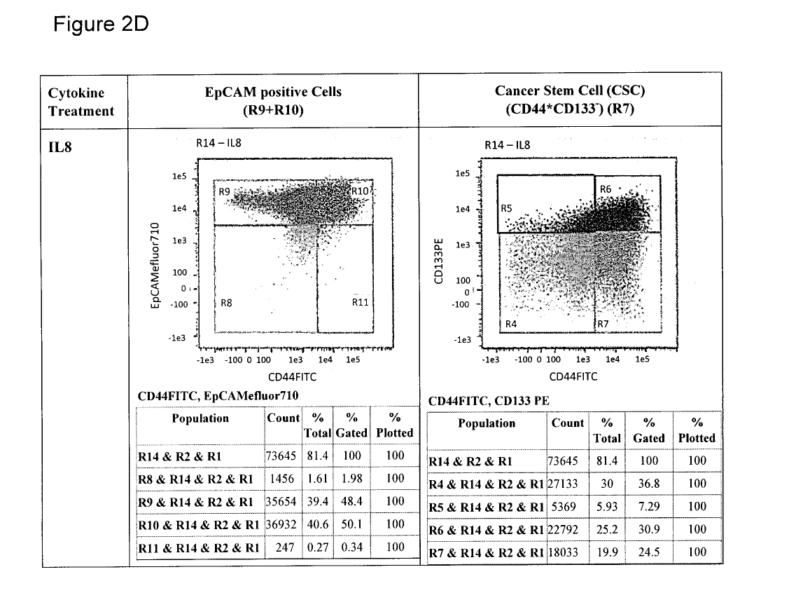

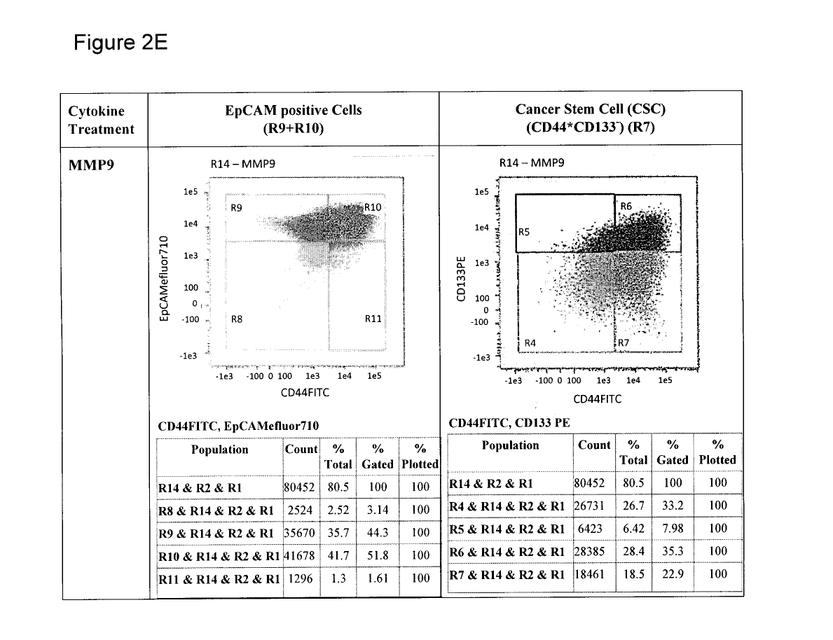

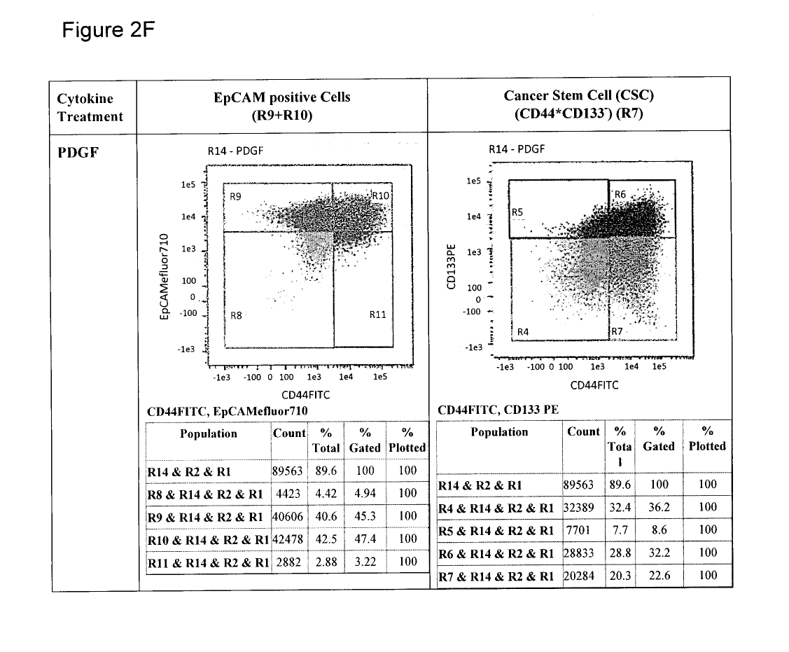

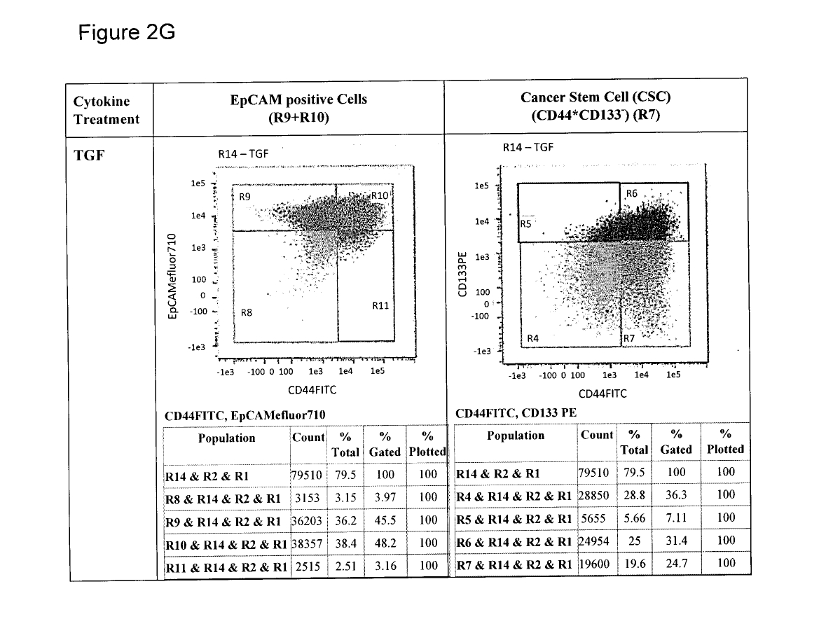

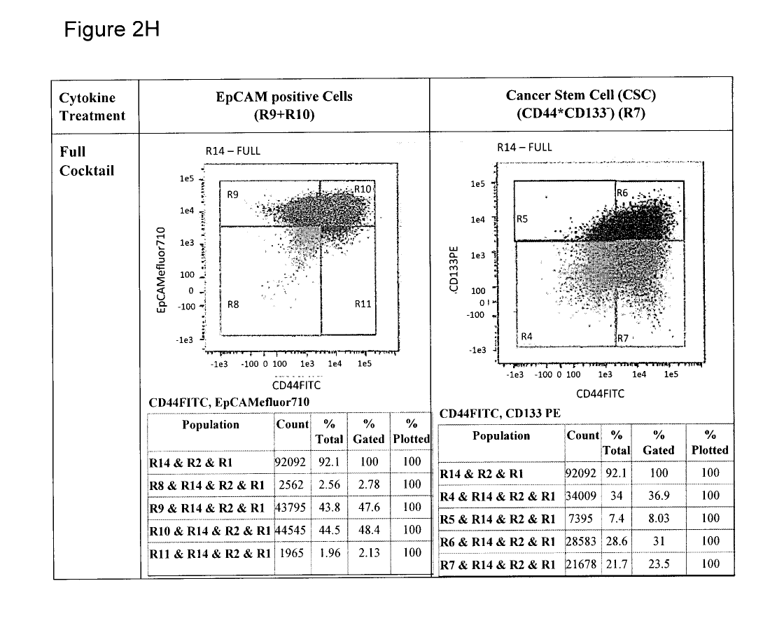

FIGS. 1A-1H, 2A-2H, and 3A-3H shows the extent of proliferation of cancer cells after incubation with cytokines. As best seen in FIGS. 2A-2H, exposure of a human cancer cell line to HGF can reduce the percentage of cancer stem cells from approximately 30% prior to exposure to less than one percent at 24 hours. This number returned to near baseline 21% at 48 hours. This demonstrates that exposure to this single agent in isolation at 1000 pg/ml fostered the maturation of an existing stem cell population towards a more differentiated phenotype. This change was transient, occurring within 24 hours and rapidly returning to baseline.

The data from the scatter plots of FIGS. 1A-1H, 2A-2H, and 3A-3H are reproduced below in Tables 1-3.

TABLE 1
Percentage of EpCAM positive Cells and Cancer Stem Cell (CSC) Subpopulation
(CD44+CD133−) after Incubation with Cytokines over a period of 0 hour
(data from scatter plots of FIGS. 1A-1H.

| | EpCAM positive Cells | | | | | Cancer Stem Cell (CSC) | | | | |
|---|---|---|---|---|---|---|---|---|---|---|
| | (R9 + R10) | | | | | (CD44⁺CD133⁻) (R7) | | | | |
| | CD44FITC, EpCAMefluor710 | | | | | CD44FITC, CD133 PE | | | | |
| Cytokine | | | % | % | % | | | % | % | % |
| Treatment | Population | Count | Total | Gated | Plotted | Population | Count | Total | Gated | Plotted |
| Control | R14 & R2 & R1 | 15149 | 81.8 | 100 | 100 | R14 & R2 & R1 | 15149 | 81.8 | 100 | 100 |
| | R8 & R14 & R2 & R1 | 629 | 3.4 | 4.15 | 100 | R4 & R14 & R2 & R1 | 8205 | 44.3 | 54.2 | 100 |
| | R9 & R14 & R2 & R1 | 8844 | 47.8 | 58.4 | 100 | R5 & R14 & R2 & R1 | 342 | 1.65 | 2.26 | 100 |
| | R10 & R14 & R2 & R1 | 5345 | 28.9 | 35.3 | 100 | R6 & R14 & R2 & R1 | 1303 | 7.04 | 8.6 | 100 |
| | R11 & R14 & R2 & R1 | 254 | 1.37 | 1.68 | 100 | R7 & R14 & R2 & R1 | 5326 | 28.8 | 35.2 | 100 |
| HGF | R14 & R2 & R1 | 36809 | 95.8 | 100 | 100 | R14 & R2 & R1 | 36809 | 95.8 | 100 | 100 |
| | R8 & R14 & R2 & R1 | 1942 | 5.06 | 5.28 | 100 | R4 & R14 & R2 & R1 | 17895 | 46.6 | 48.6 | 100 |
| | R9 & R14 & R2 & R1 | 19542 | 50.9 | 53.1 | 100 | R5 & R14 & R2 & R1 | 1327 | 3.45 | 3.61 | 100 |
| | R10 & R14 & R2 & R1 | 14368 | 37.4 | 39 | 100 | R6 & R14 & R2 & R1 | 5232 | 13.6 | 14.2 | 100 |
| | R11 & R14 & R2 & R1 | 795 | 2.07 | 2.16 | 100 | R7 & R14 & R2 & R1 | 12424 | 32.3 | 33.8 | 100 |
| IL6 | R14 & R2 & R1 | 41245 | 92.8 | 100 | 100 | R14 & R2 & R1 | 41245 | 92.8 | 100 | 100 |
| | R8 & R14 & R2 & R1 | 2124 | 4.78 | 5.15 | 100 | R4 & R14 & R2 & R1 | 20055 | 45.1 | 48.6 | 100 |
| | R9 & R14 & R2 & R1 | 21871 | 49.2 | 53 | 100 | R5 & R14 & R2 & R1 | 1437 | 3.23 | 3.48 | 100 |
| | R10 & R14 & R2 & R1 | 16066 | 36.1 | 39 | 100 | R6 & R14 & R2 & R1 | 5720 | 12.9 | 13.9 | 100 |
| | R11 & R14 & R2 & R1 | 998 | 2.24 | 2.42 | 100 | R7 & R14 & R2 & R1 | 14101 | 31.7 | 34.2 | 100 |
| IL8 | R14 & R2 & R1 | 22606 | 83.7 | 100 | 100 | R14 & R2 & R1 | 22606 | 83.7 | 100 | 100 |
| | R8 & R14 & R2 & R1 | 867 | 3.21 | 3.84 | 100 | R4 & R14 & R2 & R1 | 11056 | 40.9 | 48.9 | 100 |
| | R9 & R14 & R2 & R1 | 12425 | 46 | 55 | 100 | R5 & R14 & R2 & R1 | 809 | 2.99 | 3.58 | 100 |
| | R10 & R14 & R2 & R1 | 8795 | 32.6 | 38.9 | 100 | R6 & R14 & R2 & R1 | 3230 | 12 | 14.3 | 100 |
| | R11 & R14 & R2 & R1 | 433 | 1.6 | 1.92 | 100 | R7 & R14 & R2 & R1 | 7565 | 28 | 33.5 | 100 |
| MMP9 | R14 & R2 & R1 | 43308 | 87.1 | 100 | 100 | R14 & R2 & R1 | 43308 | 87.1 | 100 | 100 |
| | R8 & R14 & R2 & R1 | 2662 | 5.35 | 6.15 | 100 | R4 & R14 & R2 & R1 | 19819 | 39.8 | 45.8 | 100 |
| | R9 & R14 & R2 & R1 | 21892 | 44 | 50.5 | 100 | R5 & R14 & R2 & R1 | 2027 | 4.08 | 4.68 | 100 |
| | R10 & R14 & R2 & R1 | 17575 | 35.3 | 40.6 | 100 | R6 & R14 & R2 & R1 | 7978 | 16 | 18.4 | 100 |
| | R11 & R14 & R2 & R1 | 992 | 1.99 | 2.29 | 100 | R7 & R14 & R2 & R1 | 13594 | 27.3 | 31.4 | 100 |
| PDGF | R14 & R2 & R1 | 17337 | 93.6 | 100 | 100 | R14 & R2 & R1 | 17337 | 93.6 | 100 | 100 |
| Beta | R8 & R14 & R2 & R1 | 856 | 4.62 | 4.94 | 100 | R4 & R14 & R2 & R1 | 8614 | 46.5 | 49.7 | 100 |
| | R9 & R14 & R2 & R1 | 9273 | 50 | 53.5 | 100 | R5 & R14 & R2 & R1 | 425 | 2.29 | 2.45 | 100 |
| | R10 & R14 & R2 & R1 | 6727 | 36.3 | 38.8 | 100 | R6 & R14 & R2 & R1 | 1847 | 9.97 | 10.7 | 100 |
| | R11 & R14 & R2 & R1 | 404 | 2.18 | 2.33 | 100 | R7 & R14 & R2 & R1 | 6474 | 34.9 | 37.3 | 100 |
| TGF Beta | R14 & R2 & R1 | 34458 | 96.3 | 100 | 100 | R14 & R2 & R1 | 34458 | 96.3 | 100 | 100 |
| | R8 & R14 & R2 & R1 | 2127 | 5.95 | 6.17 | 100 | R4 & R14 & R2 & R1 | 16083 | 45 | 46.7 | 100 |
| | R9 & R14 & R2 & R1 | 17496 | 48.9 | 50.8 | 100 | R5 & R14 & R2 & R1 | 1354 | 3.79 | 3.93 | 100 |
| | R10 & R14 & R2 & R1 | 13546 | 37.9 | 39.3 | 100 | R6 & R14 & R2 & R1 | 5606 | 15.7 | 16.3 | 100 |
| | R11 & R14 & R2 & R1 | 1144 | 3.2 | 3.32 | 100 | R7 & R14 & R2 & R1 | 11499 | 32.1 | 33.4 | 100 |
| Full | R14 & R2 & R1 | 24316 | 92.4 | 100 | 100 | R14 & R2 & R1 | 24316 | 92.4 | 100 | 100 |
| Cocktail | R8 & R14 & R2 & R1 | 1262 | 4.8 | 5.19 | 100 | R4 & R14 & R2 & R1 | 11025 | 41.9 | 45.3 | 100 |
| | R9 & R14 & R2 & R1 | 12587 | 47.8 | 51.8 | 100 | R5 & R14 & R2 & R1 | 1135 | 4.31 | 4.67 | 100 |
| | R10 & R14 & R2 & R1 | 9806 | 37.3 | 40.3 | 100 | R6 & R14 & R2 & R1 | 4158 | 15.8 | 17.1 | 100 |
| | R11 & R14 & R2 & R1 | 567 | 2.15 | 2.33 | 100 | R7 & R14 & R2 & R1 | 8064 | 30.6 | 33.2 | 100 |

TABLE 2
Percentage of EpCAM positive Cells and Cancer Stem Cell (CSC) Subpopulation
(CD44⁺CD133−) after incubation with Cytokines over a period of 24 h (data from FIGS. 2A-2H)

| | EpCAM positive Cells | | | | | Cancer Stem Cell (CSC) | | | | |
|---|---|---|---|---|---|---|---|---|---|---|
| | (R9 + R10) | | | | | (CD44*CD133⁻) (R7) | | | | |
| | CD44FITC, EpCAMefluor710 | | | | | CD44FITC, CD133 PE | | | | |
| Cytokine | | | % | % | % | | | % | % | % |
| Treatment | Population | Count | Total | Gated | Plotted | Population | Count | Total | Gated | Plotted |
| Control | R14 & R2 & R1 | 7454 | 79.2 | 100 | 100 | R14 & R2 & R1 | 7454 | 79.2 | 100 | 100 |
| | R8 & R14 & R2 & R1 | 1868 | 19.8 | 25.1 | 100 | R4 & R14 & R2 & R1 | 3677 | 39.1 | 49.3 | 100 |
| | R9 & R14 & R2 & R1 | 2631 | 27.9 | 35.3 | 100 | R5 & R14 & R2 & R1 | 454 | 4.82 | 6.09 | 100 |
| | R10 & R14 & R2 & R1 | 1916 | 20.4 | 25.7 | 100 | R6 & R14 & R2 & R1 | 1496 | 15.9 | 20.1 | 100 |
| | R11 & R14 & R2 & R1 | 1135 | 12.1 | 15.2 | 100 | R7 & R14 & R2 & R1 | 1801 | 19.1 | 24.2 | 100 |
| HGF | R14 & R2 & R1 | 85906 | 85.9 | 100 | 100 | R14 & R2 & R1 | 85906 | 85.9 | 100 | 100 |
| | R8 & R14 & R2 & R1 | 85697 | 85.7 | 99.8 | 100 | R4 & R14 & R2 & R1 | 84737 | 84.7 | 98.6 | 100 |
| | R9 & R14 & R2 & R1 | 17 | 0.02 | 0.02 | 100 | R5 & R14 & R2 & R1 | 1 | 0 | 0 | 100 |
| | R10 & R14 & R2 & R1 | 20 | 0.02 | 0.02 | 173 | R6 & R14 & R2 & R1 | 62 | 0.06 | 0.07 | 100 |
| | R11 & R14 & R2 & R1 | 173 | 0.17 | 0.2 | 100 | R7 & R14 & R2 & R1 | 870 | 0.87 | 1.01 | 100 |
| IL6 | R14 & R2 & R1 | 74623 | 81.4 | 100 | 100 | R14 & R2 & R1 | 74623 | 81.4 | 100 | 100 |
| | R8 & R14 & R2 & R1 | 3995 | 4.36 | 5.35 | 100 | R4 & R14 & R2 & R1 | 30637 | 33.4 | 41.1 | 100 |
| | R9 & R14 & R2 & R1 | 36113 | 39.4 | 48.4 | 100 | R5 & R14 & R2 & R1 | 5742 | 6.27 | 7.69 | 100 |
| | R10 & R14 & R2 & R1 | 33712 | 36.8 | 45.2 | 100 | R6 & R14 & R2 & R1 | 21459 | 23.4 | 28.8 | 100 |
| | R11 & R14 & R2 & R1 | 1448 | 1.58 | 1.94 | 100 | R7 & R14 & R2 & R1 | 16609 | 18.1 | 22.3 | 100 |
| IL8 | R14 & R2 & R1 | 73645 | 81.4 | 100 | 100 | R14 & R2 & R1 | 73645 | 81.4 | 100 | 100 |
| | R8 & R14 & R2 & R1 | 1456 | 1.61 | 1.98 | 100 | R4 & R14 & R2 & R1 | 27133 | 30 | 36.8 | 100 |
| | R9 & R14 & R2 & R1 | 35654 | 39.4 | 48.4 | 100 | R5 & R14 & R2 & R1 | 5369 | 5.93 | 7.29 | 100 |
| | R10 & R14 & R2 & R1 | 36932 | 40.6 | 50.1 | 100 | R6 & R14 & R2 & R1 | 22792 | 25.2 | 30.9 | 100 |
| | R11 & R14 & R2 & R1 | 247 | 0.27 | 0.34 | 100 | R7 & R14 & R2 & R1 | 18033 | 19.9 | 24.5 | 100 |
| MMP9 | R14 & R2 & R1 | 80452 | 80.5 | 100 | 100 | R14 & R2 & R1 | 80452 | 80.5 | 100 | 100 |
| | R8 & R14 & R2 & R1 | 2524 | 2.52 | 3.14 | 100 | R4 & R14 & R2 & R1 | 26731 | 26.7 | 33.2 | 100 |
| | R9 & R14 & R2 & R1 | 35670 | 35.7 | 44.3 | 100 | R5 & R14 & R2 & R1 | 6423 | 6.42 | 7.98 | 100 |
| | R10 & R14 & R2 & R1 | 11678 | 41.7 | 51.8 | 100 | R6 & R14 & R2 & R1 | 28385 | 28.4 | 35.3 | 100 |
| | R11 & R14 & R2 & R1 | 1296 | 1.3 | 1.61 | 100 | R7 & R14 & R2 & R1 | 18461 | 18.5 | 22.9 | 100 |
| PDGF | R14 & R2 & R1 | 89563 | 89.6 | 100 | 100 | R14 & R2 & R1 | 89563 | 89.6 | 100 | 100 |
| | R8 & R14 & R2 & R1 | 4423 | 4.42 | 4.94 | 100 | R4 & R14 & R2 & R1 | 32389 | 32.4 | 36.2 | 100 |
| | R9 & R14 & R2 & R1 | 40606 | 40.6 | 45.3 | 100 | R5 & R14 & R2 & R1 | 7701 | 7.7 | 8.6 | 100 |
| | R10 & R14 & R2 & R1 | 42478 | 42.5 | 47.4 | 100 | R6 & R14 & R2 & R1 | 28833 | 28.8 | 32.2 | 100 |
| | R11 & R14 & R2 & R1 | 2882 | 2.88 | 3.22 | 100 | R7 & R14 & R2 & R1 | 20284 | 20.3 | 22.6 | 100 |
| TGF | R14 & R2 & R1 | 79510 | 79.5 | 100 | 100 | R14 & R2 & R1 | 79510 | 79.5 | 100 | 100 |
| | R8 & R14 & R2 & R1 | 3153 | 3.15 | 3.97 | 100 | R4 & R14 & R2 & R1 | 28850 | 28.8 | 36.3 | 100 |
| | R9 & R14 & R2 & R1 | 36203 | 36.2 | 45.5 | 100 | R5 & R14 & R2 & R1 | 5655 | 5.66 | 7.11 | 100 |
| | R10 & R14 & R2 & R1 | 38357 | 38.4 | 48.2 | 100 | R6 & R14 & R2 & R1 | 24954 | 25 | 31.4 | 100 |
| | R11 & R14 & R2 & R1 | 2515 | 2.51 | 3.16 | 100 | R7 & R14 & R2 & R1 | 19600 | 19.6 | 24.7 | 100 |
| Full | R14 & R2 & R1 | 92092 | 92.1 | 100 | 100 | R14 & R2 & R1 | 92092 | 92.1 | 100 | 100 |
| Cocktail | R8 & R14 & R2 & R1 | 2562 | 2.56 | 2.78 | 100 | R4 & R14 & R2 & R1 | 34009 | 34 | 36.9 | 100 |
| | R9 & R14 & R2 & R1 | 43795 | 43.8 | 47.6 | 100 | R5 & R14 & R2 & R1 | 7395 | 7.4 | 8.03 | 100 |
| | R10 & R14 & R2 & R1 | 44545 | 44.5 | 48.4 | 100 | R6 & R14 & R2 & R1 | 28583 | 28.6 | 31 | 100 |
| | R11 & R14 & R2 & R1 | 1965 | 1.96 | 2.13 | 100 | R7 & R14 & R2 & R1 | 21678 | 21.7 | 23.5 | 100 |

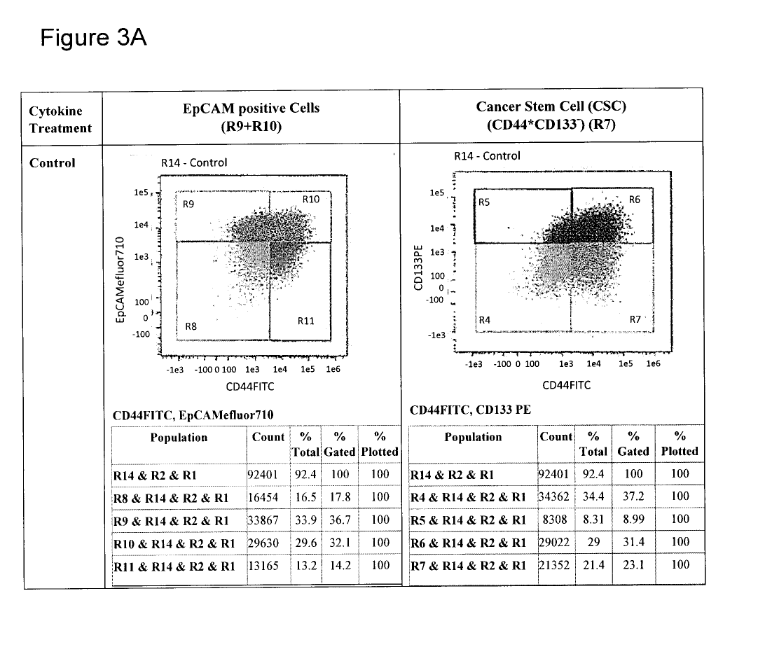

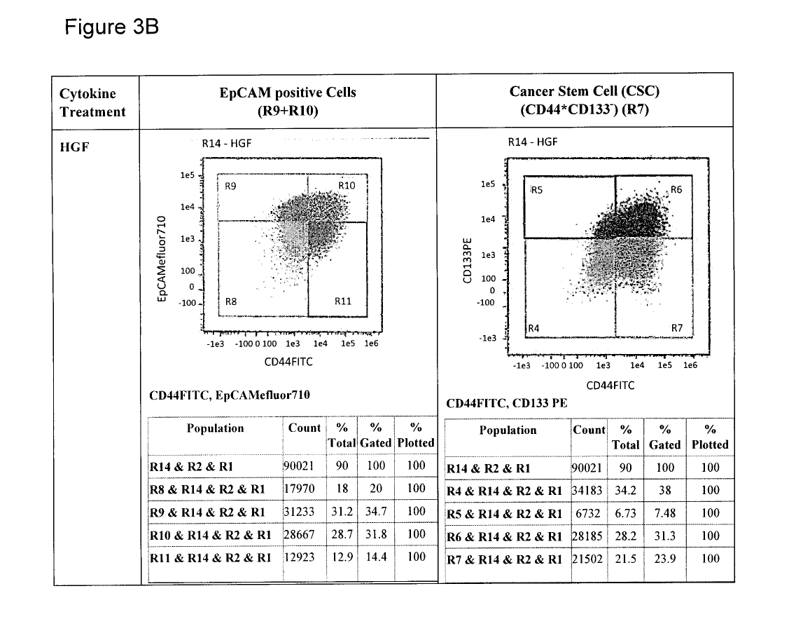

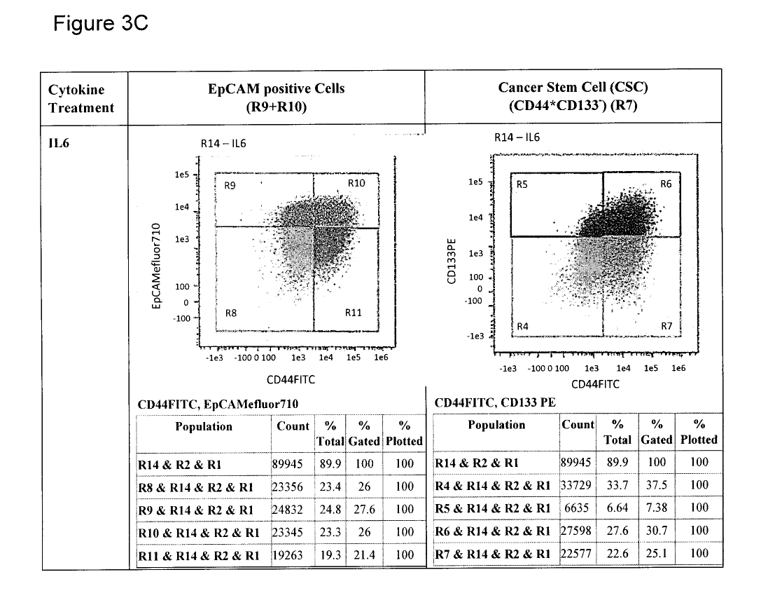

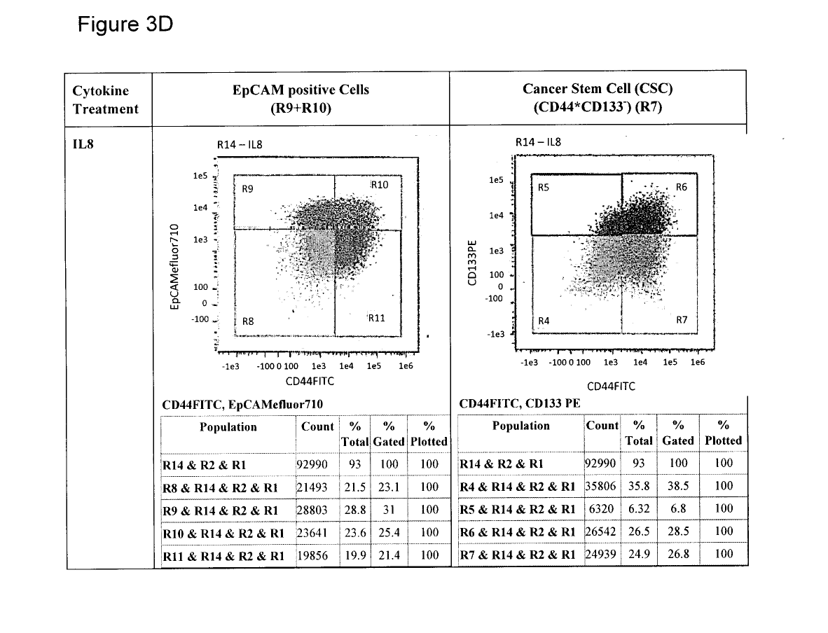

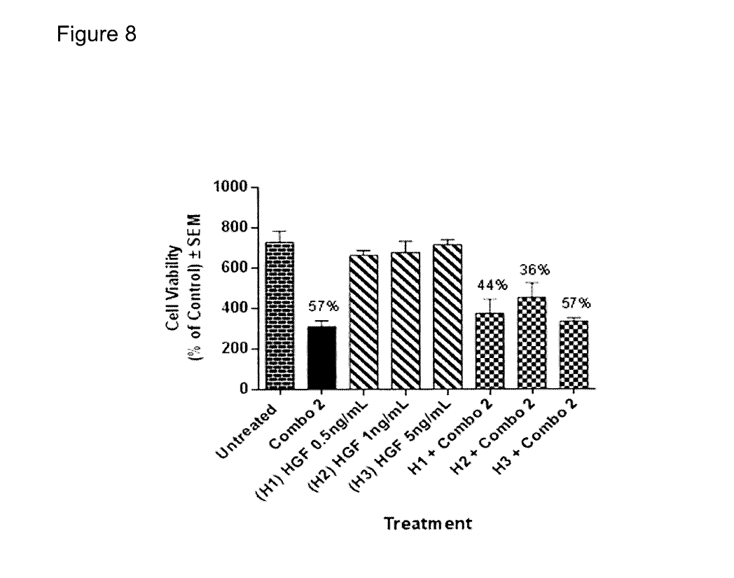

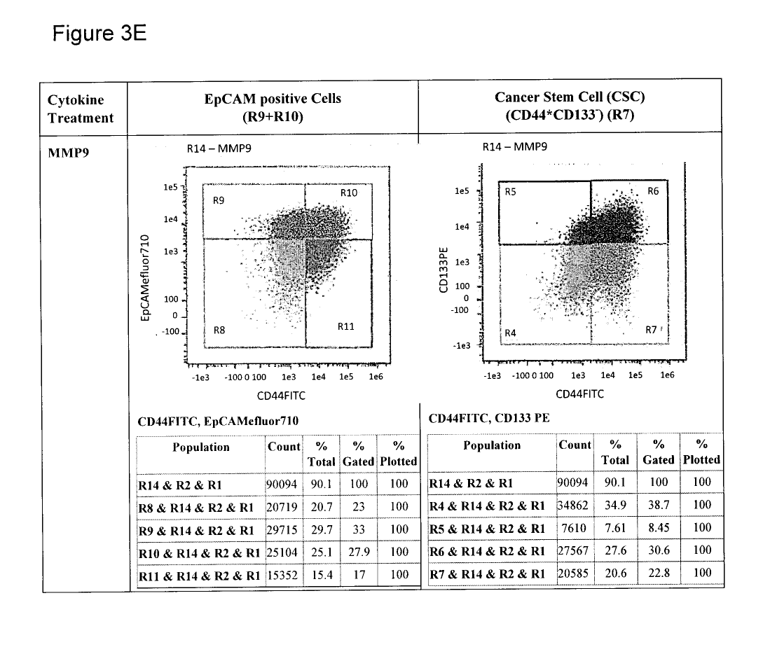

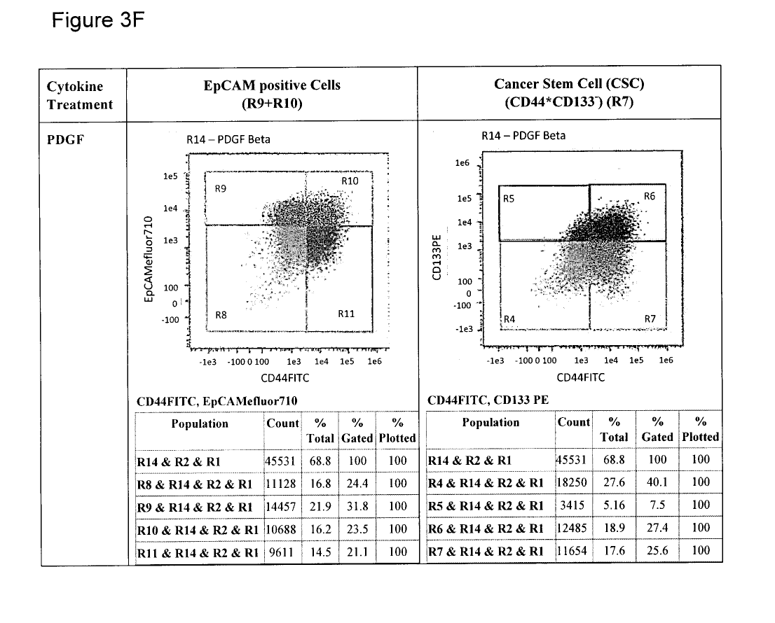

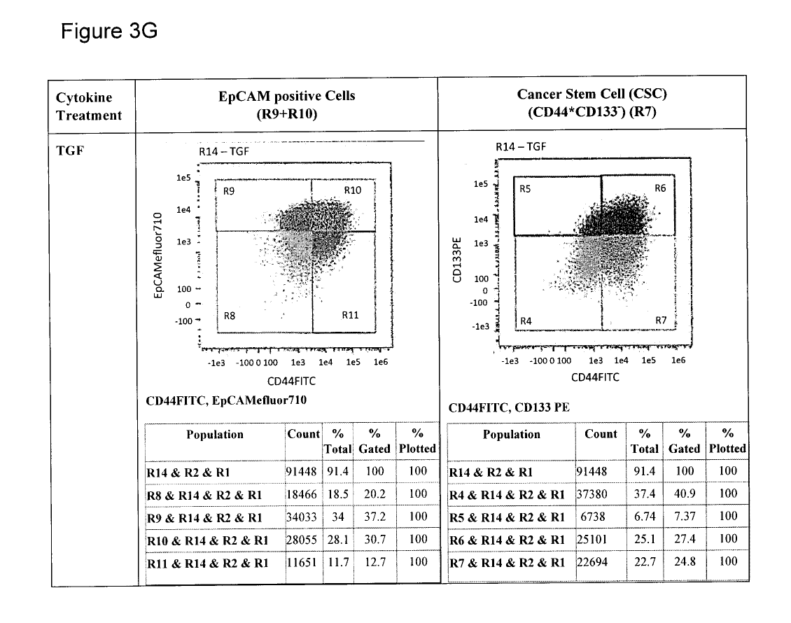

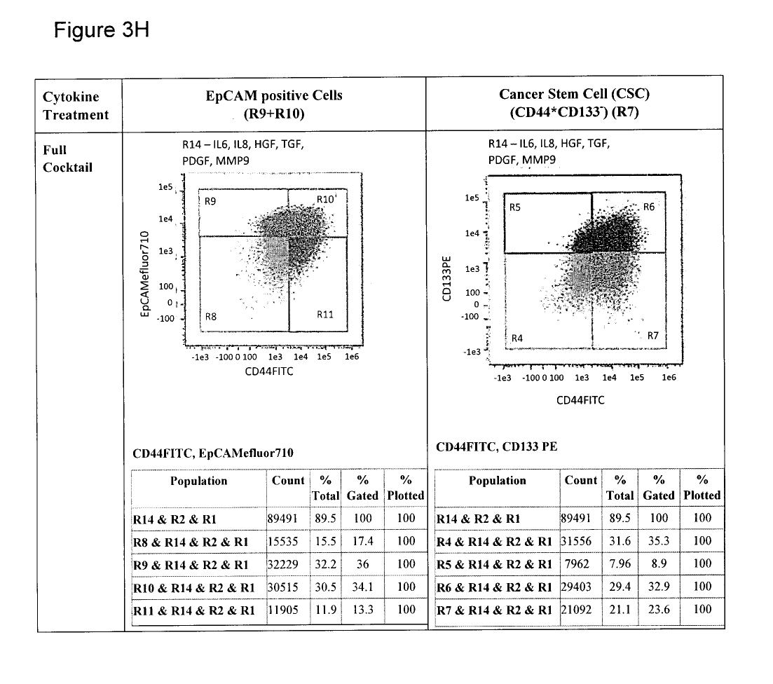

TABLE 3
Percentage of EpCAM positive Cells and Cancer Stem Cell (CSC) Subpopulation
(CD44⁺CD133−) after incubation with Cytokines over a period of 48 h (data from FIGS. 3A-3H)

| | EpCAM positive Cells | | | | | Cancer Stem Cell (CSC) | | | | |
|---|---|---|---|---|---|---|---|---|---|---|
| | (R9 + R10) | | | | | (CD44*CD133⁻) (R7) | | | | |
| | CD44FITC, EpCAMefluor710 | | | | | CD44FITC, CD133 PE | | | | |
| Cytokine | | | % | % | % | | | % | % | % |
| Treatment | Population | Count | Total | Gated | Plotted | Population | Count | Total | Gated | Plotted |
| Control | R14 & R2 & R1 | 92401 | 92.4 | 100 | 100 | R14 & R2 & R1 | 92401 | 92.4 | 100 | 100 |
| | R8 & R14 & R2 & R1 | 16454 | 16.5 | 17.8 | 100 | R4 & R14 & R2 & R1 | 34362 | 34.4 | 37.2 | 100 |
| | R9 & R14 & R2 & R1 | 33867 | 33.9 | 36.7 | 100 | R5 & R14 & R2 & R1 | 8308 | 8.31 | 8.99 | 100 |
| | R10 & R14 & R2 & R1 | 29630 | 29.6 | 32.1 | 100 | R6 & R14 & R2 & R1 | 29022 | 29 | 31.4 | 100 |
| | R11 & R14 & R2 & R1 | 13165 | 13.2 | 14.2 | 100 | R7 & R14 & R2 & R1 | 21352 | 21.4 | 23.1 | 100 |
| HGF | R14 & R2 & R1 | 90021 | 90 | 100 | 100 | R14 & R2 & R1 | 90021 | 90 | 100 | 100 |
| | R8 & R14 & R2 & R1 | 17970 | 18 | 20 | 100 | R4 & R14 & R2 & R1 | 34183 | 34.2 | 38 | 100 |
| | R9 & R14 & R2 & R1 | 31233 | 31.2 | 34.7 | 100 | R5 & R14 & R2 & R1 | 6732 | 6.73 | 7.48 | 100 |
| | R10 & R14 & R2 & R1 | 28667 | 28.7 | 31.8 | 100 | R6 & R14 & R2 & R1 | 28185 | 28.2 | 31.3 | 100 |
| | R11 & R14 & R2 & R1 | 12923 | 12.9 | 14.4 | 100 | R7 & R14 & R2 & R1 | 21502 | 21.5 | 23.9 | 100 |
| IL6 | R14 & R2 & R1 | 89945 | 89.9 | 100 | 100 | R14 & R2 & R1 | 89945 | 89.9 | 100 | 100 |
| | R8 & R14 & R2 & R1 | 23356 | 23.4 | 26 | 100 | R4 & R14 & R2 & R1 | 33729 | 33.7 | 37.5 | 100 |
| | R9 & R14 & R2 & R1 | 24832 | 24.8 | 27.6 | 100 | R5 & R14 & R2 & R1 | 6635 | 6.64 | 7.38 | 100 |
| | R10 & R14 & R2 & R1 | 23345 | 23.3 | 26 | 100 | R6 & R14 & R2 & R1 | 27598 | 27.6 | 30.7 | 100 |
| | R11 & R14 & R2 & R1 | 19263 | 19.3 | 21.4 | 100 | R7 & R14 & R2 & R1 | 22577 | 22.6 | 25.1 | 100 |
| IL8 | R14 & R2 & R1 | 92990 | 93 | 100 | 100 | R14 & R2 & R1 | 92990 | 93 | 100 | 100 |
| | R8 & R14 & R2 & R1 | 21493 | 21.5 | 23.1 | 100 | R4 & R14 & R2 & R1 | 35806 | 35.8 | 38.5 | 100 |
| | R9 & R14 & R2 & R1 | 28803 | 28.8 | 31 | 100 | R5 & R14 & R2 & R1 | 6320 | 6.32 | 6.8 | 100 |
| | R10 & R14 & R2 & R1 | 23641 | 23.6 | 25.4 | 100 | R6 & R14 & R2 & R1 | 26542 | 26.5 | 28.5 | 100 |
| | R11 & R14 & R2 & R1 | 19856 | 19.9 | 21.4 | 100 | R7 & R14 & R2 & R1 | 24939 | 24.9 | 26.8 | 100 |
| MMP9 | R14 & R2 & R1 | 90094 | 90.1 | 100 | 100 | R14 & R2 & R1 | 90094 | 90.1 | 100 | 100 |
| | R8 & R14 & R2 & R1 | 20719 | 20.7 | 23 | 100 | R4 & R14 & R2 & R1 | 34862 | 34.9 | 38.7 | 100 |
| | R9 & R14 & R2 & R1 | 29715 | 29.7 | 33 | 100 | R5 & R14 & R2 & R1 | 7610 | 7.61 | 8.45 | 100 |
| | R10 & R14 & R2 & R1 | 25104 | 25.1 | 27.9 | 100 | R6 & R14 & R2 & R1 | 27567 | 27.6 | 30.6 | 100 |
| | R11 & R14 & R2 & R1 | 15352 | 15.4 | 17 | 100 | R7 & R14 & R2 & R1 | 20585 | 20.6 | 22.8 | 100 |
| PDGF | R14 & R2 & R1 | 45531 | 68.8 | 100 | 100 | R14 & R2 & R1 | 45531 | 68.8 | 100 | 100 |
| | R8 & R14 & R2 & R1 | 11128 | 16.8 | 24.4 | 100 | R4 & R14 & R2 & R1 | 18250 | 27.6 | 40.1 | 100 |
| | R9 & R14 & R2 & R1 | 14457 | 21.9 | 31.8 | 100 | R5 & R14 & R2 & R1 | 3415 | 5.16 | 7.5 | 100 |
| | R10 & R14 & R2 & R1 | 10688 | 16.2 | 23.5 | 100 | R6 & R14 & R2 & R1 | 12485 | 18.9 | 27.4 | 100 |
| | R11 & R14 & R2 & R1 | 9611 | 14.5 | 21.1 | 100 | R7 & R14 & R2 & R1 | 11654 | 17.6 | 25.6 | 100 |
| TGF | R14 & R2 & R1 | 91448 | 91.4 | 100 | 100 | R14 & R2 & R1 | 91448 | 91.4 | 100 | 100 |
| | R8 & R14 & R2 & R1 | 18466 | 18.5 | 20.2 | 100 | R4 & R14 & R2 & R1 | 37380 | 37.4 | 40.9 | 100 |
| | R9 & R14 & R2 & R1 | 34033 | 34 | 37.2 | 100 | R5 & R14 & R2 & R1 | 6738 | 6.74 | 7.37 | 100 |
| | R10 & R14 & R2 & R1 | 28055 | 28.1 | 30.7 | 100 | R6 & R14 & R2 & R1 | 25101 | 25.1 | 27.4 | 100 |
| | R11 & R14 & R2 & R1 | 11651 | 11.7 | 12.7 | 100 | R7 & R14 & R2 & R1 | 22694 | 22.7 | 24.8 | 100 |
| Full | R14 & R2 & R1 | 89491 | 89.5 | 100 | 100 | R14 & R2 & R1 | 89491 | 89.5 | 100 | 100 |
| Cocktail | R8 & R14 & R2 & R1 | 15535 | 15.5 | 17.4 | 100 | R4 & R14 & R2 & R1 | 31556 | 31.6 | 35.3 | 100 |
| | R9 & R14 & R2 & R1 | 32229 | 32.2 | 36 | 100 | R5 & R14 & R2 & R1 | 7962 | 7.96 | 8.9 | 100 |
| | R10 & R14 & R2 & R1 | 30515 | 30.5 | 34.1 | 100 | R6 & R14 & R2 & R1 | 29403 | 29.4 | 32.9 | 100 |
| | R11 & R14 & R2 & R1 | 11905 | 11.9 | 13.3 | 100 | R7 & R14 & R2 & R1 | 21092 | 21.1 | 23.6 | 100 |

Based on the above data of potentiating the effectiveness of chemotherapy by exposing cancer cells to specific cytokines such as HGF and or Il-6 within a time limited fashion, the mechanism behind this ability to sensitize cancer cells may not simply be triggering cellular proliferation. Specific cytokine exposures can transiently cause a cancer cell population to differentiate away from a more stem cell enriched, mesenchymal phenotype towards a more differentiated, epithelial cell population more sensitive to the effects of anti-cancer therapies (see Tables 1-3). In these tables, exposure to various cytokines over 24-48 hours revealed distinct phenotypic changes in the SW620 colorectal cancer cell line studied and triggered enhanced cytotoxicity of cancer treatments such as chemotherapy.

In particular, exposure to a control population of SW620 colorectal cancer cell line to HGF at a concentration of 10 ng per ml for 24 hours caused the control putative stem cell population (CD44+/CD133−) to drop from 33% at baseline (see Table 1, R7 region, CD 44 positive/CD133 negative) to 1% (see Table 2, R7, region CD 44 positive/CD133 negative). This drop was transient, as exposure to the same cell line for 48 hours of HGF at 10 ng per ml transitioned the phenotypic distribution back towards a stem cell enriched phenotype with 23% putative stem cells (see Table 3, R7 region, CD 44 positive/CD133 negative).

### Immunotherapy

Tumor cells interact closely with the stromal cells, immune cells, and extracellular matrix that is part of the tumor microenvironment (TME). This TME can play an important role in limiting the ability of immune cells to detect and eradicate cancer cells. Within this TME a specific transcriptome, also referred to as an innate anti-PD-1 resistance signature or IPRES signature, can predict for resistance to immunotherapy (Hugo et al., 2016). Some of the genes that are upregulated and are part of the IPRES signature include mesenchymal transition genes such as AXL, WNT5A, LOXL2, TWIST2, FAP), angiogenesis genes, wound healing genes, as well as immunosuppressive genes.

The present inventor has identified that this characteristic transcriptome, or IPRES signature, will be amenable to therapeutic manipulation using adjuvant therapies that can reverse this molecular signature and underlying biology towards a transcriptome or phenotype more receptive to effective checkpoint inhibitor blockade. In other words, it is possible to therapeutically manipulate a cancerous tumor prior to the use of checkpoint inhibitors, making primary resistant tumors receptive to immunotherapy and reverse the acquired resistance to immunotherapy that often develops in patients whom initially respond to immunotherapy.

For similar reasons as explained above, exposure to HGF and/or IL-6 within the time frame discussed in the Examples above would also potentiate the effectiveness of immunotherapy with checkpoint inhibitors by triggering differentiation of a cancer cell population towards a more differentiated, and less mesenchymal, phenotype. The time limited nature of this exposure is illustrated in Table 3, whereby exposure to this cytokine for 48 hour is starting to trigger a tumor cell dedifferentiation or a phenotypic shift backwards to a more immature, mesenchymal/stem cell phenotype. This experiment demonstrates the critical nature of time exposure in sensitizing a tumor to the effects of immunotherapy. By exposing cancerous tumors in vivo to HGF alone, or in combination with IL-6, for 12 up to 48 hours prior to the administration of immunotherapy, sensitization to the effects of immunotherapy can occur and more patients could be treated more effectively with these checkpoint inhibitors. In some embodiments, the combination of cytokines is HGF, or HGF and Il-6. In various embodiments, the combination of cytokines includes, at least one, at least two, at least three, at least four at least five, at least six, at least seven, or all of, Hepatocyte Growth Factor (HGF), IL-6, PGE-2, MCP-1, MMP-9, TGF-beta, PDGF-BB, and PGF. Various combinations of the other cytokines can also be used. Accordingly, acquired resistance to checkpoint inhibitors can be overcome using cytokine sensitization as described.

### Example 2

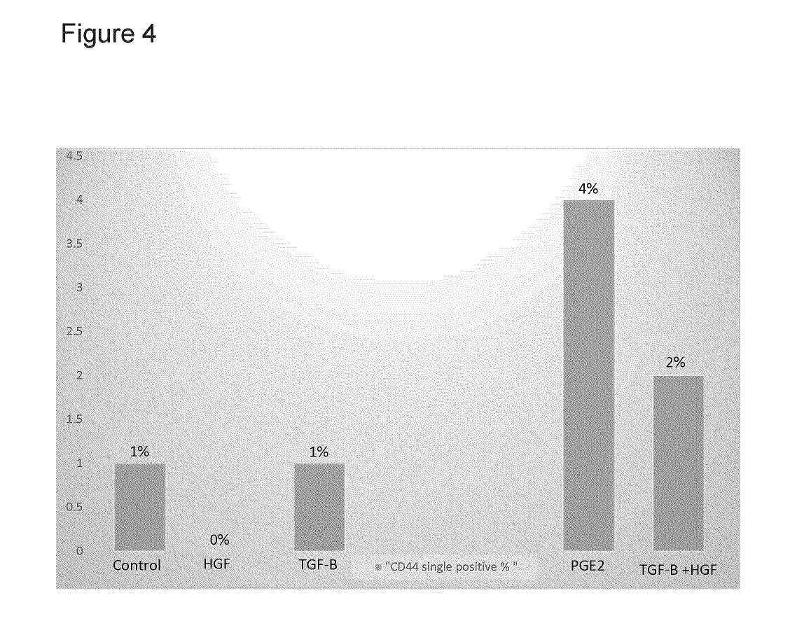

The present inventor then performed experiments to determine if exposure to HGF would improve the effectiveness of chemotherapy treatment, based on a prediction that this stimulus, by fostering differentiation of the stem cell subpopulation, would render these cells more susceptible to chemotherapy. As seen in the data presented in FIG. 4, the log kill ratio after exposure to HGF was 100%, with no viable cancer stem cells remaining. Control cell populations not exposed to HGF had a viable cancer cell population remaining, with a viable cancer stem cell population surviving.

### Example 3

The present inventor has demonstrated an upregulation and/or an enhanced proliferation of cancer stem cells when the cancer cells were treated with a combination of HGF and IL-6.

In the following experiments various concentrations of hepatocyte growth factor (HGF) and Interleukin 6 (IL-6), used singly and together, were used to sensitize cancer cell lines to cytotoxic chemotherapy. In these experiments a combination anti-cancer treatment of paclitaxel with aspirin and oseltamivir phosphate (OP) were used. Cell lines treated with the combination treatment (COMBO) alone were compared with those treated with the combination treatment after exposure to cytokines.

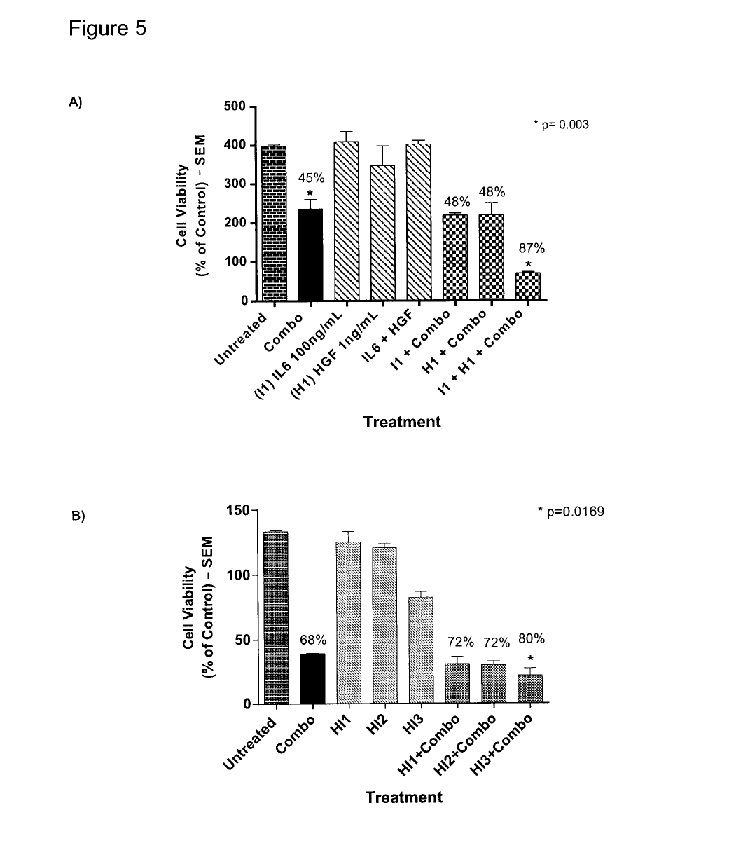

In FIG. 5, exposure to IL-6 and HGF for a 12 hour period sensitized cancer cell lines to subsequent combination treatment with a statistically significant increase in cancer cell death as compared to combination treatment by itself. The results showed 87% reduction in cell viability after sensitization versus 45% reduction in cell viability with combination treatment only. P-value in FIG. 5 was calculated based on comparison between Combination Treatment vs. Pretreatment Followed by Combination Treatment. Significance of Combo condition is compared to Untreated. Statistical comparison of Untreated vs HI3 shows that cells might be sensitive to Tmx present in media.

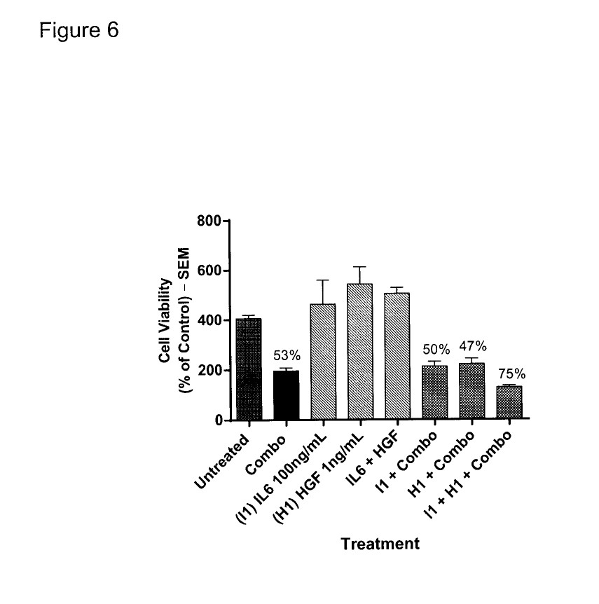

In FIG. 6, administration of Il-6 and HGF for 24 hours also showed a sensitizing effect as this combination was shown to lead to a statistically significant increase in cell death with subsequent combination treatment as opposed to treatment with the combination treatment in isolation. The results showed 75% reduction in cell viability after sensitization versus 53 percent reduction in cell viability with combination treatment only.

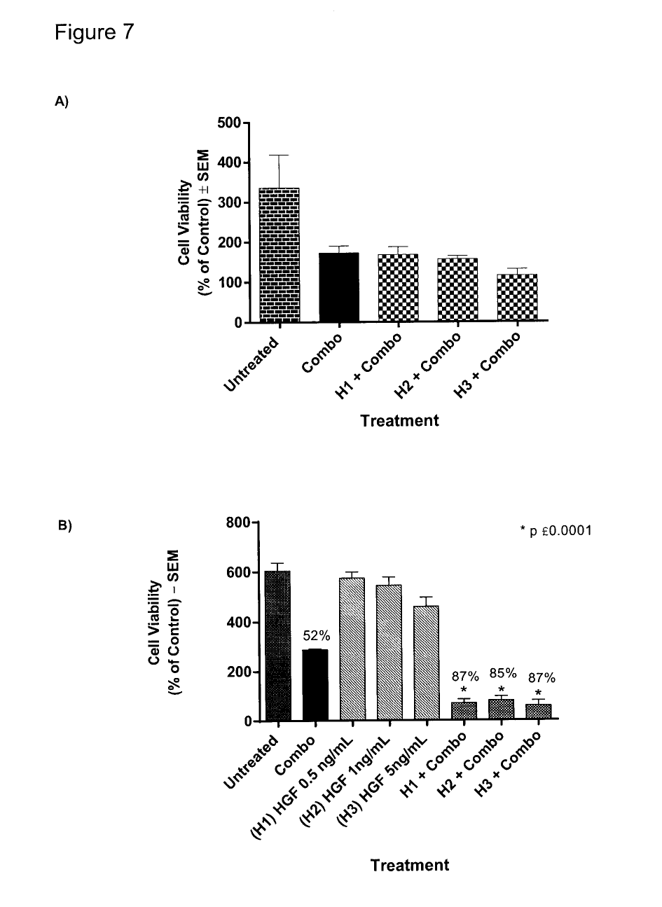

In FIG. 7, exposing human pancreatic cancer cell lines (PANC-1) cells to HGF for 48 hours at various concentrations sensitized cancer cells to subsequent combination treatment. The results showed 87% reduction in cell viability with sensitization followed by combination treatment compared to 52% reduction in cell viability with combination treatment alone. P-value in FIG. 7 was calculated based on comparison between Combination Treatment vs. Pretreatment Followed by Combination Treatment.

Furthermore, exposure to HGF alone for 24 hours at a concentration of 10 ng per ml followed by treatment with CPT-11 in SW 620 cell lines was able to kill cancer stem cells effectively as well, given that there were no remaining cancer stem cells after this sensitization (FIG. 4 0% CD44+/CD133−).

The ability of these cytokines, alone or in combination, to sensitize cancer cells to subsequent cancer therapies is time dependent. Exposure to cytokines more than 72 hours, more than 60 hours and, in one embodiment, more than 48 hours before commencing chemotherapy may prove detrimental to the effectiveness of treatments such as chemotherapy by, for example, fostering enrichment for a cancer stem cell phenotype as explained above.

Specifically, in FIG. 8 it is shown that exposure to HGF for 72 hours not only did not sensitize cancer cells to the combination treatment, but at the exemplified dose made the combination treatment less effective, indicating that this difference in efficacy is secondary to the time sensitive nature of the treatment approach. Thus, exposure to this particular cytokine, for longer than 48 hours can increase the likelihood that the cancer cell population is transitioning over to a more stem cell enriched phenotype, rendering the cells less sensitive to the effects of cytotoxic therapies. Other cytokines such as HGF, IL-6, PGE-2, MCP-1, MMP-9, TGF-beta, PDGF-BB, and PGF, the up-regulation of which is associated with stem cell enrichment should also be administered in this time limited manner to avoid a stem cell enriched phenotype that is more resistant to the chemotherapy treatment. Moreover, it is proposed that in order for this treatment methodology to be effective, care must be taken to limit cytokine exposure to less than 48-72 hours. Approximately, the time point where cytokines cease to be sensitizing and more likely to be triggering stem cell enrichment is likely within 48-72 hours of chemotherapy treatment.

All reference documents and/or patent documents listed herein are incorporated herein by reference to the extent that they do not contradict with the subject matter of this application.

### REFERENCES

1. Sharma, P and Allison J. P. The future of immune checkpoint therapy, Science 348 (6230) (2015) 56-61.
2. Hugo, W et al. Genomic and transcriptomic features of response to anti-PD-1 therapy in metastatic melanoma, Cell 165 (1) (2016) 35-44.
3. O'Shea, Leah K et al. “Therapeutic Targeting of Neu1 Sialidase with Oseltamivir Phosphate (Tamiflu®) Disables Cancer Cell Survival in Human Pancreatic Cancer with Acquired Chemoresistance.” OncoTargets and therapy 7 (2014): 117-134.
4. Ohm, J. E et al. VEGF inhibits T-cell development and may contribute to tumor-induced immune suppression, Blood 101 (12) (2003) 4878-4886.
5. Sawaoka, H et al. Cyclooxygenase inhibitors suppress angiogenesis and reduce tumor growth in vivo. Lab Investigation 79 (12) (1999):1469-77.
6. Mendelsohn J and Baselga J. Status of epidermal growth factor receptor antagonists in the biology and treatment of cancer. J Clin Oncol. 2003; 21(14):2787-2799.
7. Peng, W. et al. Loss of PTEN promotes resistance to T cell-mediated immunotherapy, Cancer Discov. 6 (2) (2016) 202-216.
8. Uddin, S. et al. Cyclooxygenase-2 inhibition inhibits PI3K/AKT kinase activity in epithelial ovarian cancer. International Journal of Cancer 126, (2010) 382-394.
9. Mani, S. A. et al. “The Epithelial-Mesenchymal Transition Generates Cells with Properties of Stem Cells.” Cell 133.4 (2008): 704-715.

1-27. (canceled)

28. A method of treating cancer comprising administering to a patient in need thereof an effective amount composition for treating cancer comprising at least one cytokine that induces the cancer cell population to proliferate and/or differentiate within 48+/−8 hours of a chemotherapy treatment.

29. The method of claim 28, wherein the at least one cytokine is administered within 24+/−8 hours of a chemotherapy treatment.

30. The method of claim 28, wherein the at least one cytokine is administered within 12+/−8 hours of a chemotherapy treatment.

31. The method of claim 28 wherein the treatment with at least one cytokine is administered prior to commencement of the chemotherapy treatment.

32. The method of claim 31, wherein the at least one cytokine is administered at most 72 to 48 hours prior to chemotherapy treatment.

33. The method of claim 31, wherein the at least one cytokine is administered 48+/−8 hours prior to chemotherapy treatment.

34. The method of claim 31, wherein the at least one cytokine is administered 24+/−8 hours prior to chemotherapy treatment.

35. The method of claim 31, wherein the at least one cytokine is administered 12+/−8 hours prior to chemotherapy treatment.

36. The method of claim 31, wherein the chemotherapy treatment is discontinued between about 48 and 96 hours following treatment with the at least one cytokine.

37. The method of claim 28, wherein the at least one cytokine is HGF.

38. The method of claim 28, wherein the at least one cytokine is HGF and Il-6.

39. The method of claim 28, wherein the at least one cytokine is one or more of Il-6, PDGF-BB, TGF-beta, and HGF.

40. The method of claim 28, wherein the chemotherapy treatment comprises a neu-1 sialidase inhibitor.

41. The method of claim 40, wherein the neu-1 sialidase inhibitor is oseltamivir phosphate.

42. The method of claim 41, wherein the chemotherapy treatment further comprises a non-steroidal anti-inflammatory drug (NSAID).

43. The method of claim 42, wherein the NSAID is aspirin.

44. The method of claim 40, wherein the chemotherapy treatment further comprises a biguanide.

45. The method of claim 44, wherein the biguanide is metformin.

46. The method of claim 28, wherein the chemotherapy treatment comprises a checkpoint inhibitor.

47. The method of claim 31 wherein administration of the at least one cytokine sensitizes the cancer cells to chemotherapy.
